(19) United States
(12) Patent Application Publication
(10) Pub. No.: US 2024/0011105 A1
(43) Pub. Date: Jan. 11, 2024

(54) ANALYSIS OF MICROBIAL FRAGMENTS IN PLASMA

(71) Applicants: The Chinese University of Hong Kong, Shatin (HK); GRAIL, Inc., Menlo Park, CA (US)

(72) Inventors: Yuk-Ming Dennis Lo, Kowloon (CN); Kwan Chee Chan, Kowloon (CN); Rossa Wai Kwun Chiu, Shatin (CN); Wai Kei Lam, Kowloon (CN); Peiyong Jiang, Tai Po (CN); Guangya Wang, Shatin (CN)

(21) Appl. No.: 17/860,577

(22) Filed: Jul. 8, 2022

(51) Int. Cl. C12Q 1/6888; G16B 30/10; C12Q 1/686; C12Q 1/6809

(52) U.S. Cl. CPC C12Q 1/6888; G16B 30/10; C12Q 1/686; C12Q 1/6809

(57) ABSTRACT

Various embodiments are directed to detecting infection-causing microbial cell-free DNA from a biological sample based on their size profiles and/or end signatures, in which the detection of infection-causing microbial DNA can be performed without no template control (NTC) samples. Embodiments can include identifying the infection-causing pathogen-derived microbial DNA based on sizes of microbial cell-free DNA molecules. Embodiments can also include identifying from the infection-causing pathogen-derived microbial DNA based on end signatures of microbial cell-free DNA molecules. Embodiments can also include applying a machine-learning algorithm to a plurality of vectors that represent end signatures of the microbial cell-free DNA molecules, to identify the infection-causing pathogen-derived microbial DNA. By detecting the infection-causing pathogen-derived microbial DNA, a level of infection for the biological sample can be predicted.

### BACKGROUND

The human microbiota, including but not limited to bacteria, DNA viruses and fungi, plays a critical role in causing infection and this represents a major threat to the health of human. Recently, a number of studies illustrated that microbiome-derived DNA fragments could be detected in the blood circulation (Han et al. Theranostics. 2020; 10:5501-5513). In the context of infection (e.g., sepsis), microbiological culture-based methods are the gold-standard tests for the identification of causative pathogens. However, these methods usually take a long time to yield the results, and many pathogens are difficult to be cultured outside the human body.

Accordingly, there is a need for a more robust, efficient, reproducible, and effective technique that can detect microbial DNA and use the detected microbial DNA to predict a level of infection in a subject.

### SUMMARY

Embodiments are directed to systems and methods for analyzing infection-causing pathogen-derived microbial cell-free DNA from a biological sample based on their size profiles and/or end signatures, in which the detection of infection-causing pathogen-derived microbial DNA can be performed by obviating the requirement of no template control (NTC) samples. By detecting the infection-causing pathogen-derived microbial DNA, a level of infection for the biological sample can be predicted. For example, the size profile or end signatures of infection-causing pathogen-derived microbial DNA can be predictive of sepsis, which is a life-threatening condition that occurs when the subject's body triggers an extreme response to an infection.

In some instances, the infection-causing pathogen-derived microbial DNA are identified based on sizes of microbial cell-free DNA molecules. The sequences can be identified by aligning sequence reads of the biological sample to one or more reference microbe genomes, in which each reference microbe genome corresponds to a particular species of microbes. Sizes of the identified microbial cell-free DNA molecules can be measured. Then, a statistical value can be derived from the measured sizes of the microbial cell-free DNA molecules. The statistical value can be compared to a cutoff value to determine a level of infection of the subject.

The infection-causing pathogen-derived microbial DNA can be identified based on end signatures of microbial cell-free DNA molecules. Sequence reads corresponding to the microbial cell-free DNA molecules can be identified by aligning sequence reads of the biological sample to one or more reference microbe genomes, in which each reference microbe genome corresponds to a particular species of microbes. From the microbial sequence reads, a set of sequence reads that include ending sequences that correspond to one or more sequence end signatures can be identified. A parameter can then be determined based on a first amount of the set of sequence reads. The parameter can be used to determine a classification of a level of infection.

In some instances, a plurality of sequence end signatures are used to identify infection-causing pathogen-derived microbial DNA in a biological sample, rather than using one or more specific sequence end signatures. A machine-learning model can process a plurality of vectors to determine whether the biological sample includes infection-causing pathogen-derived microbial DNA from one or more microbe species, in which each vector of the plurality of vectors represents a respective sequence end signature.

Other embodiments are directed to systems, portable consumer devices, and computer readable media associated with methods described herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows examples for end motifs according to some embodiments of the present disclosure.

FIG. 2 illustrates fragmentomic features based analysis for plasma microbial cell-free DNA, according to some embodiments.

FIGS. 3A-B illustrate an overview of microbial cell-free DNA analysis based on sizes and end signatures, according to some embodiments.

FIG. 4 shows a graph that identifies a correlation between procalcitonin (PCT) level and overall microbial cell-free DNA abundance, according to some embodiments.

FIGS. 5A-C show a set of diagrams that identify difference in fragment size between infection-causing pathogen-derived microbial DNA and contaminant DNA, according to some embodiments.

FIG. 6 is a flowchart illustrating a method of determining a level of infection in a biological sample based on size characteristics of microbial cell-free DNA, according to some embodiments.

FIGS. 7A-C show diagrams that identify different preferences in 1-mer end signatures for infection-causing pathogen-derived microbial DNA and contaminant DNA, according to some embodiments.

FIGS. 8A-C show diagrams that identify different preference in 2-mer end signatures for infection-causing pathogen-derived microbial DNA and contaminant DNA, according to some embodiments.

FIGS. 9A-D show diagrams that identify a comparison of cases with and without infection regarding the overall end signatures of microbial DNA, according to some embodiments.

FIG. 10 is a flowchart illustrating a method for determining a level of infection in a biological sample based on sequence end signatures, according to some embodiments.

FIGS. 11A-B show diagrams that identify comparisons of the preference regarding end motifs of microbial cell-free DNA and contaminant microbial DNA in a public dataset, according to some embodiments.

FIGS. 12A-B show diagrams that identify differences in end signatures of contaminant Pseudomonas-derived DNA and pathogenic Pseudomonas-derived DNA, according to some embodiments.

FIGS. 13A-C show diagrams identifying 4-mer end motif signatures that can be applied to distinguish septic cases from non-septic cases, according to some embodiments.

FIG. 14 is a flowchart illustrating a method for using machine-learning techniques to determine a level of infection in a biological sample, according to some embodiments.

FIGS. 15A-B show diagrams that identify end signatures of microbial DNA fragments in pregnant subjects, according to some embodiments.

FIG. 16 illustrates a measurement system according to an embodiment of the present invention.

FIG. 17 illustrates example subsystems that implement a measurement system according to an embodiment of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells). The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” may be used to refer to a tissue from which a cell-free nucleic acid originates.

The terms “sample”, “biological sample,” or “patient sample” refer to any sample that is taken from a subject, pregnant or non-pregnant, suspected of having an infection and contains one or more nucleic acid molecule(s) of interest. The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells. As part of an analysis of a biological sample, at least 1,000 cell-free DNA molecules can be analyzed. As other examples, at least 10,000 or 50,000 or 100,000 or 500,000 or 1,000,000 or 5,000,000 cell-free DNA molecules, or more, can be analyzed.

The terms “control”, “control sample”, “background sample,” “reference”, “reference sample”, “normal”, and “normal sample” may be interchangeably used to generally describe a sample that does not have a particular condition, or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

The phrase “healthy,” as used herein, generally refers to a subject possessing good health. Such a subject demonstrates an absence of any malignant or non-malignant disease. A “healthy individual” may have other diseases or conditions, unrelated to the condition being assayed, that may normally not be considered “healthy”.

A “sequence read” refers to a string of nucleotides sequenced from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed.

The term “fragment” (e.g., a DNA fragment) refers to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins.

The term “infection-causing pathogen-derived microbial DNA” refers to DNA molecules originating from one or more species of microbes known to cause infection in organisms (e.g., humans).

The term “contaminant DNA” refers to foreign DNA molecules that that do not originate from a biological sample of a subject. For example, the contaminant DNA can be unintentionally added into the biological sample when reagents (e.g. adapters, linkers, and PCR primers that attach to DNA molecules of the biological sample as part of cloning or amplification process) are added to generate a sequencing library. Contaminant DNA can originate from various sources, e.g., molecular biology grade water, DNA extraction kits and laboratory environment. Contaminant DNA can be considered as non-pathogenic DNA.

The terms “size profile” and “size distribution” generally relate to the sizes of DNA fragments in a biological sample. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range.

A “cutting site” can refer to a location that nucleic acid, e.g., DNA, is cut by a nuclease, thereby resulting in a nucleic acid, e.g., DNA, fragment.

An “ending position” or “end position” (or just “end) can refer to the genomic coordinate or genomic identity or nucleotide identity of the outermost base, i.e., at the extremities, of a cell-free DNA molecule, e.g., plasma DNA molecule. The end position can correspond to either end of a DNA molecule. In this manner, if one refers to a start and end of a DNA molecule, both may correspond to an ending position. In practice, one end position is the genomic coordinate or the nucleotide identity of the outermost base on one extremity of a cell-free DNA molecule that is detected or determined by an analytical method, such as but not limited to massively parallel sequencing or next-generation sequencing, single molecule sequencing, double- or single-stranded DNA sequencing library preparation protocols, polymerase chain reaction (PCR), or microarray. Such in vitro techniques may alter the true in vivo physical end(s) of the cell-free DNA molecules. Thus, each detectable end may represent the biologically true end or the end is one or more nucleotides inwards or one or more nucleotides extended from the original end of the molecule e.g., 5′ blunting and 3′ filling of overhangs of non-blunt-ended double stranded DNA molecules by the Klenow fragment. The genomic identity or genomic coordinate of the end position may be derived from results of alignment of sequence reads to a human reference genome, e.g., hg38. It may be derived from a catalog of indices or codes that represent the original coordinates of the human genome. It may refer to a position or nucleotide identity on a cell-free DNA molecule that is read by but not limited to target-specific probes, mini-sequencing, DNA amplification.

The term “genomic position” can refer to a nucleotide position in a polynucleotide (e.g., a gene, a plasmid, a nucleic acid fragment, a microbial DNA fragment). The term “genomic position” is not limited to nucleotide positions within a genome (e.g., the haploid set of chromosomes in a gamete or microorganism, or in each cell of a multicellular organism).

The term “ending sequence” refers to an end of a sequence read. The ending sequence can correspond to the outermost N bases of the fragment, e.g., 2-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” of “sequence end signature” may refer to a short, recurring pattern of bases in nucleic acid fragments (e.g., cell-free DNA fragments). A sequence motif can occur at an end of a fragment, and thus be part of or include an ending sequence. An “end motif” can refer to a sequence motif for an ending sequence that preferentially occurs at ends of nucleic acid, e.g., DNA, fragments, potentially for DNA molecules originating from pathogenic microbes. An end motif may also occur just before or just after ends of a fragment, thereby still corresponding to an ending sequence.

A “relative frequency” may refer to a proportion (e.g., a percentage, fraction, or concentration). In particular, a relative frequency of a particular end motif (e.g., CCGA) can provide a proportion of cell-free DNA fragments that are associated with the end motif CCGA, e.g., by having an ending sequence of CCGA.

The term “relative abundance” may generally refer to a ratio of a first amount of nucleic acid fragments having a particular characteristic (e.g., a specified length, ending at one or more specified coordinates/ending positions, or aligning to a particular region of the genome) to a second amount nucleic acid fragments having a particular characteristic (e.g., a specified length, ending at one or more specified coordinates/ending positions, or aligning to a particular region of the genome). In one example, relative abundance may refer to a ratio of the number of DNA fragments ending at a first set of genomic positions to the number of DNA fragments ending at a second set of genomic positions. In some aspects, “relative abundance” may correspond to a type of separation value that relates an amount (one value) of cell-free DNA molecules ending within one window of genomic positions to an amount (other value) of cell-free DNA molecules ending within another window of genomic positions. The two windows may overlap, but may be of different sizes. In other implementations, the two windows may not overlap. Further, the windows may be of a width of one nucleotide, and therefore be equivalent to one genomic position.

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1). As further examples, the levels of classification can correspond to a fractional concentration or a value for a characteristic, e.g., of a sample or of a target tissue type.

The terms “cutoff” “threshold,” or reference level can refer to a predetermined number used in an operation. A threshold or reference value may be a value above or below which a particular classification applies, e.g., a classification of a condition, such as whether a subject has a condition or a severity of the condition. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level) can be used to determine reference levels to discriminate between the different conditions and/or classifications of a condition (e.g., whether the subject has the condition). Any of these terms can be used in any of these contexts.

A “level of infection” can refer to the presence or absence, or an amount of pathogens present in a biological sample. For example, the level of infection can indicate a number of sequence reads associated with pathogens (e.g., reads per million) that are obtained from a plasma sample of a subject. The presence of pathogens can indicative the amount, degree, or severity of infection associated with an organism. In some instances, the amount, degree, or severity of infections is predicted based on the amount of infective microorganisms in the biological sample. The level of infection may be a number or other indicia, such as symbols, alphabet letters, and colors. The level may be zero. The level of infection can be used in various ways. The infection can be predictive of a pathology associated with the organism, as well as a type of tissue at which the infection has occurred. The infection can be caused by various types of pathogens, including bacteria and other microorganisms. The level of infection can also indicate a type of infection, such as tuberculosis, anthrax, tetanus, leptospirosis, pneumonia, cholera, botulism, and Pseudomonas infection. In some instances, the level of infection refers to a condition relating to an organism's response to microbes, including sepsis, bacteremia, and septicemia.

The term “assay” generally refers to a technique for determining a property of a nucleic acid. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity, and their relative usefulness as a diagnostic tool can be measured using ROC-AUC statistics.

The term “true positive” (TP) can refer to subjects having a condition. True positive generally refers to subjects that have an infection (e.g., sepsis). True positive generally refers to subjects having a condition, and are identified as having the condition by an assay or method of the present disclosure.

The term “true negative” (TN) can refer to subjects that do not have a condition or do not have a detectable condition. True negative generally refers to subjects that do not have a disease or a detectable disease, including infection. True negative generally refers to subjects that do not have a condition or do not have a detectable condition, or are identified as not having the condition by an assay or method of the present disclosure.

The term “false positive” (FP) can refer to subjects not having a condition. False positive generally refers to subjects not having an infection. The term false positive generally refers to subjects not having a condition, but are identified as having the condition by an assay or method of the present disclosure.

The term “false negative” (FN) can refer to subjects that have a condition. False negative generally refers to subjects that have an infection. The term false negative generally refers to subjects that have a condition, but are identified as not having the condition by an assay or method of the present disclosure.

The terms “sensitivity” or “true positive rate” (TPR) can refer to the number of true positives divided by the sum of the number of true positives and false negatives. Sensitivity may characterize the ability of an assay or method to correctly identify a proportion of the population that truly has a condition. For example, sensitivity may characterize the ability of a method to correctly identify the number of subjects within a population having an infection. In another example, sensitivity may characterize the ability of a method to correctly identify one or more markers indicative of an infection.

The terms “specificity” or “true negative rate” (TNR) can refer to the number of true negatives divided by the sum of the number of true negatives and false positives. Specificity may characterize the ability of an assay or method to correctly identify a proportion of the population that truly does not have a condition. For example, specificity may characterize the ability of a method to correctly identify the number of subjects within a population not having an infection. In another example, specificity may characterize the ability of a method to correctly identify one or more markers indicative of an infection.

The term “ROC” or “ROC curve” can refer to the receiver operator characteristic curve. The ROC curve can be a graphical representation of the performance of a binary classifier system. For any given method, an ROC curve may be generated by plotting the sensitivity against the specificity at various threshold settings. The sensitivity and specificity of a method for predicting a level of infection in a subject may be determined at various concentrations of infection-causing pathogen-derived microbial DNA in the plasma sample of the subject. Furthermore, provided at least one of the three parameters (e.g., sensitivity, specificity, and the threshold setting), and ROC curve may determine the value or expected value for any unknown parameter. The unknown parameter may be determined using a curve fitted to the ROC curve. The term “AUC” or “ROC AUC” generally refers to the area under a receiver operator characteristic curve. This metric can provide a measure of diagnostic utility of a method, taking into account both the sensitivity and specificity of the method. Generally, ROC-AUC ranges from 0.5 to 1.0, where a value closer to 0.5 indicates the method has limited diagnostic utility (e.g., lower sensitivity and/or specificity) and a value closer to 1.0 indicates the method has greater diagnostic utility (e.g., higher sensitivity and/or specificity). See, e.g., Pepe et al, “Limitations of the Odds Ratio in Gauging the Performance of a Diagnostic, Prognostic, or Screening Marker,” Am. J. Epidemiol 2004, 159 (9): 882-890, which is entirely incorporated herein by reference. Additional approaches for characterizing diagnostic utility using likelihood functions, odds ratios, information theory, predictive values, calibration (including goodness-of-fit), and reclassification measurements are summarized according to Cook, “Use and Misuse of the Receiver Operating Characteristic Curve in Risk Prediction,” Circulation 2007, 115: 928-935, which is entirely incorporated herein by reference.

“Negative predictive value” or “NPV” may be calculated by TN/(TN+FN) or the true negative fraction of all negative test results. Negative predictive value is inherently impacted by the prevalence of a condition in a population and pre-test probability of the population intended to be tested. “Positive predictive value” or “PPV” may be calculated by TP/(TP+FP) or the true positive fraction of all positive test results. It is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which is entirely incorporated herein by reference.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and in some versions within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. It is also to be understood that the endpoints of the range provided are included in the range. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Next generation sequencing (NGS)-based microbial cell-free DNA testing would potentially enable non-invasive diagnosis of different kinds of infectious diseases and determine the pathogen profiles, providing useful information to guide antibiotic administration (Gu et al. Nat Med. 2021; 27:115-124). Besides, plasma microbial DNA analysis holds promise for the detection of antibiotic resistance genetic markers. In addition, microbial nucleic acids are fragmented in blood circulation (Han et al. Theranostics. 2020; 10:5501-5513; Burnham et al. Sci Rep. 2016; 6:27859). Fragmentation processes of microbial cell-free DNA may involve different DNA nucleases, in which the DNA fragments may potentially exhibit various fragmentomic features (Serpas et al. Proc Natl Acad Sci USA. 2019; 116:641-649; Han et al. Am J Hum Genet. 2020; 106:202-14).

The microbial cell-free DNA is often present in plasma at a low abundance. In the NGS-based microbial cell-free DNA testing, contamination from the environment can become a major issue. The contamination could occur during sample collection, sample processing (such as DNA extraction), and sequencing. The contamination can limit the accuracy of data interpretation. For example, a plasma sample with infection-causing pathogen-derived microbial DNA can be contaminated when reagents are added to generate a sequencing library. In another example, contamination could occur based on the environment at which the biological samples are sequenced.

To address the potential contamination, decontamination analysis can be performed. One technique for decontamination includes subjecting one or more no-template control (NTC) sample(s), together with clinical samples, to all the laboratory steps for sequencing analysis. The NTC sample includes only solution that is not supposed to include any DNA molecules, but with the same elution volume for DNA extraction in clinical samples, such as but not limited to molecular biology grade water, phosphate-buffered saline etc. NTC samples can include microbial DNA molecules, but these microbial DNA molecules are generally considered as non-pathogenic, contaminant DNA. This is because the microbial DNA molecules in NTC samples are residual microbial DNA that are introduced when one or more reagents are added into the NTC samples. The clinical samples would also likely include the contaminant DNA for the same reason. To distinguish the contaminant DNA from infection-causing pathogen-derived microbial DNA, the DNA sequences obtained from the NTC samples can be compared with the DNA sequences obtained from a clinical sample of a subject known to have an infection (e.g., pneumonia). Since both samples include contaminant DNA due to the added reagents, for the traditional approach, the microbial species in clinical samples that show higher DNA abundance (above pre-defined cutoffs) compared with NTC samples would be considered as true signals for having infection-causing pathogen-derived microbial DNA. The infection-causing pathogen-derived DNA (also referred to as “microbial DNA of pathogens”) in a biological sample can be confirmed with a microbiology test.

Another technique can include detecting infection-causing pathogen-derived microbial DNA based on abundance of DNA molecules that align to one or more reference microbe genomes. For example, a clinical sample can be considered as having infection-causing pathogen-derived microbial DNA if the amount of microbial DNA molecules exceeds a predetermined threshold.

However, the above techniques have drawbacks. First, the comparison using NTC samples assumes that all DNA molecules in NTC samples are non-pathogenic. But, the NTC samples can sometimes include infection-causing pathogen-derived microbial DNA. As a result, using NTC samples to filter contaminant DNA could trigger false negatives by erroneously removing genuinely pathogenic microbial species that are incidentally present in NTC samples. Second, using abundance of DNA molecules often does not produce accurate results in cell-free samples, because setting the thresholds is very difficult in view of the low abundance of microbial cell-free DNA in plasma samples.

To address the above deficiencies, the present techniques can detect infection-causing pathogen-derived microbial cell-free DNA from a biological sample based on their size profiles and/or end signature, in which the detection of infection-causing pathogen-derived DNA can be performed without the NTC samples. By detecting the infection-causing pathogen-derived microbial DNA, a level of infection for the biological sample can be predicted. For example, the size profile or end signatures of infection-causing pathogen-derived microbial DNA can be predictive of sepsis, which is a life-threatening condition that occurs when the subject's body triggers an extreme response to an infection.

In some instances, the infection-causing pathogen-derived microbial DNA are identified based on sizes of microbial cell-free DNA molecules. The microbial cell-free DNA molecules can be identified by aligning sequence reads of the biological sample to one or more reference microbe genomes, in which each reference microbe genome corresponds to a particular species of microbes. Sizes of the identified microbial cell-free DNA molecules can be measured. Then, a statistical value (e.g., mean, median) can be derived from the measured sizes of the microbial cell-free DNA molecules. The statistical value can be compared to a cutoff value to determine a level of infection of the subject. For example, if the statistical value exceeds the cutoff value, it can be determined that the microbial cell-free DNA molecules include infection-causing pathogen-derived microbial DNA.

The infection-causing pathogen-derived microbial DNA can be identified based on end signatures of microbial cell-free DNA molecules. Sequence reads corresponding to the microbial cell-free DNA molecules can be identified by aligning sequence reads of the biological sample to one or more reference microbe genomes, in which each reference microbe genome corresponds to a particular species of microbes. From the microbial sequence reads, a set of sequence reads that include ending sequences that correspond to one or more sequence end signatures (e.g., C, GG, CCCA) can be identified. A parameter can then be determined based on a first amount of the set of sequence reads. The parameter can be used to determine a classification of a level of infection.

The parameter can indicate whether the DNA molecules in the cell-free biological sample include infection-causing pathogen-derived microbial DNA. For example, the parameter can be an observed frequency of the microbial DNA molecules. In some instances, the parameter corresponds to a ratio of observed to expected frequency of the microbial DNA molecules having the end signature (the “O/E ratio”). The infection-causing pathogen-derived microbial DNA of the biological sample would show different characteristics from those of the contaminant DNA of the background sample. In some instances, sequences having ending sequences that correspond to a plurality of end signatures can be processed together using a machine-learning model to predict the level of infection.

In some instances, a plurality of sequence end signatures are used to identify infection-causing pathogen-derived microbial DNA in a biological sample, rather than using one or more specific sequence end signatures. A machine-learning model (e.g., a support vector machine) can process a plurality of vectors to determine whether the biological sample includes infection-causing pathogen-derived microbial DNA from one or more microbe species, in which each vector of the plurality of vectors represents a respective sequence end signature (e.g., CCCA).

As a result, we developed new approaches to model fragmentomic features for differentiating between microbial DNA fragments from pathogens and contaminant microbial DNA, without the requirement of NTC samples. Further, the signals of size profiles and end signatures of infection-causing pathogen-derived microbial cell-free DNA can be readily detected in cell-free samples that include contaminant DNA. These signature-based approaches could be used to enhance the signal-to-noise ratio for diagnosis, screening, and monitoring of diseases associated with microbes.

Certain techniques described herein thus improve predicting infection (e.g., sepsis) of a subject by accurately detecting infection-causing pathogen-derived cell-free microbial DNA from a biological sample of the subject. As explained above, using the NTC samples can trigger false negatives by classifying certain infection-causing pathogen-derived DNA as contaminant DNA. Further, the NTC samples may have different amounts of various contaminants based on the reagents that are added to the NTC samples, further complicating the determination as to whether DNA molecules in NTC samples correspond to infection-causing pathogen-derived DNA. In effect, accurate prediction of a level of infection using NTC-based techniques becomes challenging and difficult.

By contrast, the present techniques obviate the need of NTC samples and use fragmentomic features (e.g., size, end signatures) of microbial cell-free DNA to detect pathogenic microbial species that may be removed when using NTC samples. The sizes and end signatures can be advantageous as they can be consistently used as features that can distinguish infection-causing pathogen-derived DNA from contaminant DNA. Analysis involving size and end signatures of microbial cell-free DNA would also save the reagents and sequencing cost, as well as minimize the information loss regarding microbial DNA fragments in plasma samples. The increased accuracy and efficiency allow an improvement in predicting infection in various subjects.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Celsius, and pressure is at or near atmospheric.

### I. Cell-Free DNA Sequence End Signatures

A sequence end signature can be considered relevant for a physiological or disease state when it has a high likelihood or probability for being detected in that physiological or pathological state. The physiological or disease state can include presence of infection-causing pathogen-derived DNA that may cause infection (e.g., sepsis) in patients. In some instances, the sequence end signatures are considered relevant for the physiological or disease state if ending sequences corresponding to the end motifs are detected at a greater frequency in subjects having the disease. Because the probability of detecting the sequence end signatures in a relevant physiological or disease state is higher, such ending sequences corresponding to sequence end signatures would be seen in more than one individual with that same physiological or disease state.

A catalog of sequence end signatures associated with particular physiological states or pathological states can be identified by comparing the cell-free DNA profiles of end motifs among individuals with different physiological or pathological states. After a catalog of cell-free DNA preferred ends is established for any physiological or pathological state, targeted or non-targeted methods can be used to detect their presence in cell-free DNA samples, e.g. plasma, or other individuals to determine a classification of the other tested individuals having a similar health, physiologic or disease state (e.g., a level of infection).

### A. Sequencing Techniques

An end motif can relate to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

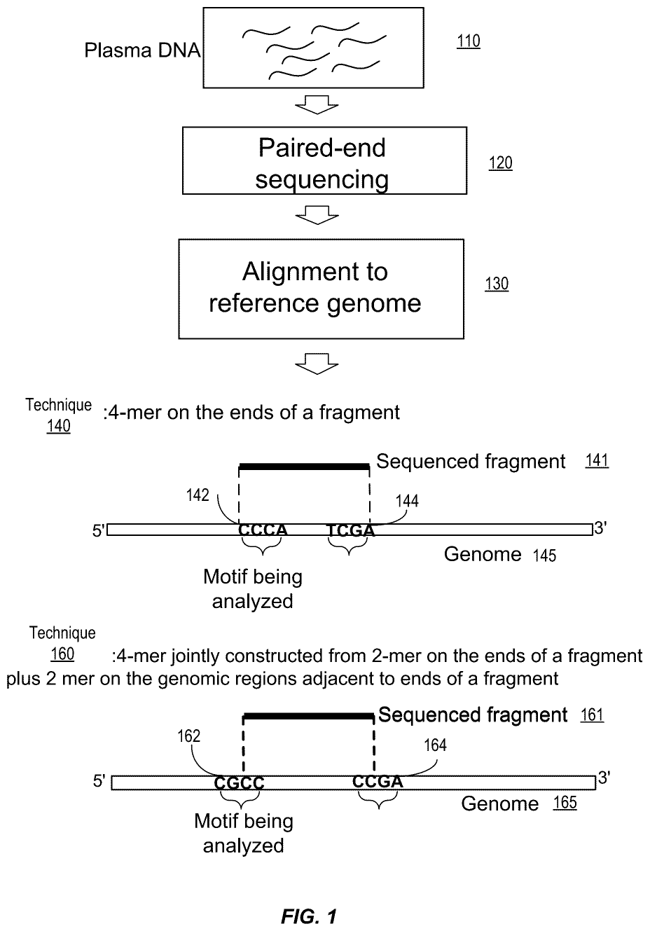

FIG. 1 shows examples for end motifs according to embodiments of the present disclosure. FIG. 1 depicts two ways to define 4-mer end motifs to be analyzed. In technique 140, the 4-mer end motifs are directly constructed from the first 4-bp sequence on each end of a plasma DNA molecule. For example, the first 4 nucleotides or the last 4 nucleotides of a sequenced fragment could be used. In technique 160, the 4-mer end motifs are jointly constructed by making use of the 2-mer sequence from the sequenced ends of fragments and the other 2-mer sequence from the genomic regions adjacent to the ends of that fragment. In other embodiments, other types of motifs can be used, e.g., 1-mer, 2-mer, 3-mer, 5-mer, 6-mer, 7-mer end motifs.

As shown in FIG. 1, cell-free DNA fragments 110 are obtained, e.g., using a purification process on a blood sample, such as by centrifuging. Besides plasma DNA fragments, other types of cell-free DNA molecules can be used, e.g., from serum, urine, saliva, and other mentions herein. In some instances, the DNA fragments may be blunt-ended.

At block 120, the DNA fragments are subjected to paired-end sequencing. In some embodiments, the paired-end sequencing can produce two sequence reads from the two ends of a DNA fragment, e.g., 30-120 bases per sequence read. These two sequence reads can form a pair of reads for the DNA fragment (molecule), where each sequence read includes an ending sequence of a respective end of the DNA fragment. In other embodiments, the entire DNA fragment can be sequenced, thereby providing a single sequence read, which includes the ending sequences of both ends of the DNA fragment.

At block 130, the sequence reads can be aligned to a reference genome. This alignment is to illustrate different ways to define a sequence motif, and may not be used in some embodiments. The alignment procedure can be performed using various software packages, such as BLAST, FASTA, Bowtie, BWA, BFAST, SHRiMP, SSAHA2, NovoAlign and SOAP.

Technique 140 shows a sequence read of a sequenced fragment 141, with an alignment to a genome 145. With the 5′ end viewed as the start, a first end motif 142 (5′-CCCA) is at the start of sequenced fragment 141. A second end motif 144 (TCGA) is at the tail of the sequenced fragment 141. In some embodiments, as the strand of double-stranded DNA has orientations, 5′ end motifs are consistently used when determining the end motif profile (i.e., reading the end motif information starting from 5′-end). For example, the second end motif 144 (TCGA) would be in silico converted to (5′-TCGA). When analyzing the end predominance of a cell-free DNA (cell-free DNA) fragments (e.g., plasma DNA), this sequence read would contribute to a C-end count for the 5′ end. Such end motifs may occur when an enzyme recognizes CCCA and then makes a cut just before the first C. If that is the case, CCCA will preferentially be at the end of the plasma DNA fragment. For TCGA, an enzyme might recognize it, and then make a cut after the A.

Technique 160 shows a sequence read of a sequenced fragment 161, with an alignment to a genome 165. With the 5′ end viewed as the start, a first end motif 162 (CGCC) has a first portion (CG) that occurs just before the start of sequenced fragment 161 and a second portion (CC) that is part of the ending sequence for the start of sequenced fragment 161. A second end motif 164 (CCGA) has a first portion (GA) that occurs just after the tail of sequenced fragment 161 and a second portion (CC) that is part of the ending sequence for the tail of sequenced fragment 161. Such end motifs may occur when an enzyme recognizes CGCC and then makes a cut just before the G and the C. If that is the case, CC will preferentially be at the end of the plasma DNA fragment with CG occurring just before it, thereby providing an end motif of CGCC. As for the second end motif 164 (CCGA), an enzyme can cut between C and G. If that is the case, CC will preferentially be at the end of the plasma DNA fragment. For technique 160, the number of bases from the adjacent genome regions and sequenced plasma DNA fragments can be varied and are not necessarily restricted to a fixed ratio, e.g., instead of 2:2, the ratio can be 2:3, 3:2, 4:4, 2:4, etc.

The higher the number of nucleotides included in the cell-free DNA end signature, the higher the specificity of the motif because the probability of having 6 bases ordered in an exact configuration in the genome is lower than the probability of having 2 bases ordered in an exact configuration in the genome. Thus, the choice of the length of the end motif can be governed by the needed sensitivity and/or specificity of the intended use application.

As the ending sequence is used to align the sequence read to the reference genome, any sequence motif determined from the ending sequence or just before/after is still determined from the ending sequence. Thus, technique 160 makes an association of an ending sequence to other bases, where the reference is used as a mechanism to make that association. A difference between techniques 140 and 160 would be to which two end motif a particular DNA fragment is assigned, which affects the particular values for the relative frequencies. But, the overall result (e.g., fractional concentration of clinically-relevant DNA, classification of a level of pathology, etc.) would not be affected by how the a DNA fragment is assigned to an end motif, as long as a consistent technique is used for the training data as used in production.

The counted numbers of DNA fragments having an ending sequence corresponding to a particular end motif may be counted (e.g., stored in an array in memory) to determine relative frequencies. As described in more detail below, a relative frequency of end motifs for cell-free DNA fragments can be analyzed. Differences in relative frequencies of end motifs have been detected for different types of tissue and for different phenotypes, e.g., different levels of pathology. The differences can be quantified by an amount of DNA fragments having specific end motifs or an overall pattern, e.g., a variance (such as entropy, also called a motif diversity score), across a set of end motifs (e.g., all possible combinations of the k-mers corresponding to the length used).

### B. Additional Techniques

Additionally or alternatively, hybridization capture of loci with high density of end motifs could be performed on the cell-free DNA samples to enrich the sample with cell-free DNA molecules with such end motifs following but not limited to detection by sequencing, microarray, or the PCR. In some instances, amplification based approaches are used to specifically amplify and enrich for the microbial nucleic acid fragments with ending sequences corresponding to the sequence end signatures, e.g. inverse PCR, rolling circle amplification. The amplification products could be identified by sequencing, microarray, fluorescent probes, gel electrophoresis and other standard approaches known to those skilled in the art.

In practice, one end position can be the genomic coordinate or the nucleotide identity of the outermost base on one extremity of a cell-free DNA molecule that is detected or determined by an analytical method, such as but not limited to massively parallel sequencing or next-generation sequencing, single molecule sequencing, double- or single-stranded DNA sequencing library preparation protocols, PCR, other enzymatic methods for DNA amplification (e.g. isothermal amplification) or microarray. Such in vitro techniques may alter the true in vivo physical end(s) of the cell-free DNA molecules. Thus, each detectable end may represent the biologically true end or the end is one or more nucleotides inwards or one or more nucleotides extended from the original end of the molecule.

For example, the Klenow fragment is used to create blunt-ended double-stranded DNA molecules during DNA sequencing library construction by blunting of the 5′ overhangs and filling in of the 3′ overhangs. Though such procedures may reveal a cell-free DNA end position that is not identical to the biological end, clinical relevance could still be established. This is because the identification of the preferred being relevant or associated with a particular physiological or pathological state could be based on the same laboratory protocols or methodological principles that would result in consistent and reproducible alterations to the cell-free DNA ends in both the calibration sample(s) and the test sample(s). A number of DNA sequencing protocols use single-stranded DNA libraries (Snyder et al Cell 2016, 164: 57-68). The ends of the sequence reads of single-stranded libraries may be more inward or extended further than the ends of double-stranded DNA libraries.

### II. Overview of Size and End Signature Analyses of Microbial Cell-Free DNA

Plasma cell-free DNA (cell-free DNA) fragmentation is associated with different DNA nucleases that have different cutting preferences (Han et al. Am J Hum Genet. 2020; 106:202-14). Hence, a set of specific cell-free DNA end signatures would be generated during such non-random cell-free DNA fragmentation. We reasoned that microbial cell-free DNA released into the blood circulation would also be subjected to digestion with various DNA nucleases. In some instances, the nucleases would include DNASE1L3 (Deoxyribonuclease 1 Like 3), DNASE1 (Deoxyribonuclease 1) and DFFB (DNA fragmentation factor subunit beta).

In contrast to microbial DNA fragments present in the blood circulation, contaminant DNA fragments from the environment (introduced from the laboratory processes, and/or present in reagents) would lack the nuclease-mediated cutting. Microbial cell-free DNA-associated fragmentomic features can be used to distinguish true infection-causing pathogen-derived DNA from contamination. Analysis of the fragmentomic features would therefore improve the signal-to-noise ratio, thus reducing false positive rate in pathogen detection in NGS-based microbial cell-free DNA testing (FIG. 1). Such fragmentomic features can include fragment sizes and/or end motifs.

### A. Fragmentomic Features of Microbial Cell-Free DNA

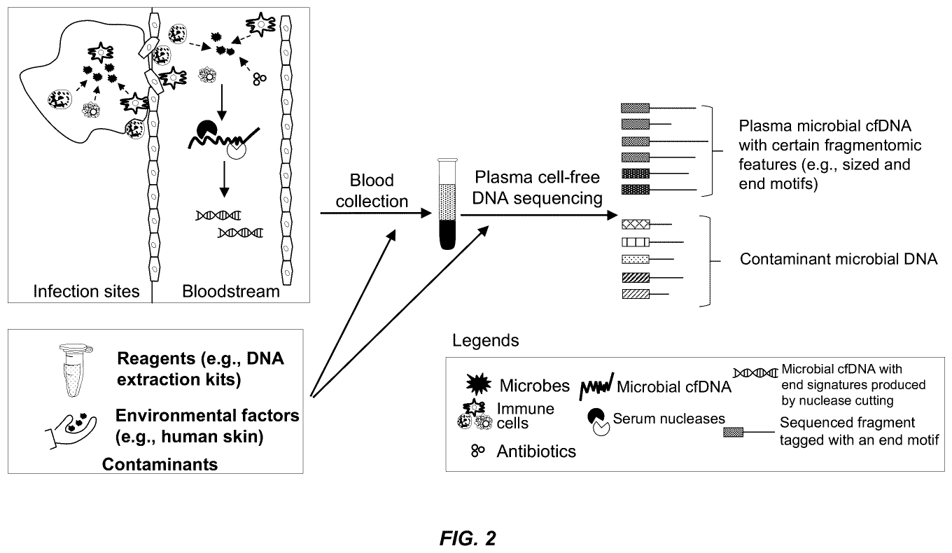

FIG. 2 illustrates fragmentomic features based analysis for plasma microbial cell-free DNA, according to some embodiments. The fragmentomic features can include sequence end signatures of DNA fragments, in which the sequence end signatures can refer to the 5′ end and/or the 3′ end. The number of nucleotides (nt) at the fragment ends used for analysis would be, for example but not limited to, 1 nt, 2 nt, 3 nt, 4 nt, 5 nt, 6 nt, 7 nt, 8 nt, 9 nt, and 10 nt or above. In some instances, nuclease-associated end motif would correspond to sites preferentially cleaved by a nuclease. In another embodiment, nuclease-associated end motifs would correspond to end motifs which are preferentially cut by one or more nucleases. In another embodiment, nuclease-associated end motifs would be defined by those end motifs which are over-represented or under-represented in disease or clinical scenarios (e.g. following transplantation), or in certain physiological states (e.g. pregnancy). In yet another embodiment, nuclease-associated end motifs could be defined by those end motifs which are over-represented or under-represented in nuclease knockout mice or other genetically modified animals.

### B. Example Analyses of Microbial Cell-Free DNA

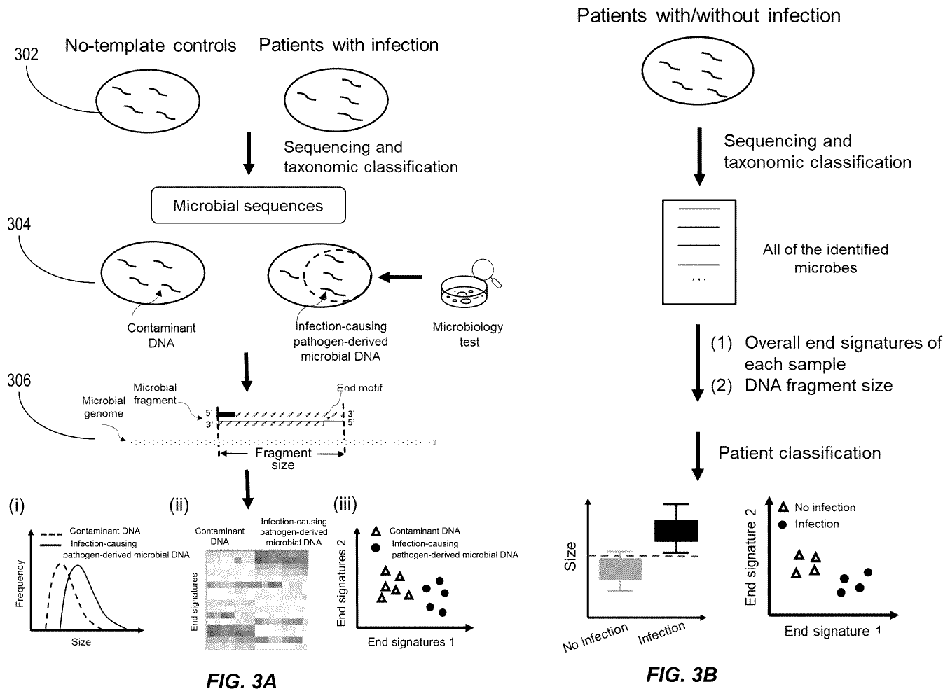

FIGS. 3A-B illustrate an overview of microbial cell-free DNA analysis based on their sizes and end signatures, according to some embodiments. FIG. 3A shows a schematic diagram of performing microbial cell-free DNA analysis using NTC samples and clinical plasma samples.

At block 302, NTC samples and clinical plasma samples were prepared and processed in parallel.

At block 304, microbial DNA sequences were determined by aligning to microbial reference genomes. The microbial sequences identified in NTC samples were referred to as contaminant DNA (i.e. dataset 1). For samples from patients with infection, the microbial sequences detected in plasma which were concordant with those microbes identified in microbiology tests (e.g. culture-based methods) were referred to as infection-causing pathogen-derived microbial DNA (i.e. dataset 2).

At block 306, fragmentomic signatures (e.g. fragment size, end motif, etc.) can be analysed in infection-causing pathogen-derived microbial DNA and contaminant DNA. Such comparison would generate fragmentomic signature-based classifiers for distinguishing infection-causing pathogen-derived microbial DNA from contaminant DNA. FIG. 3B shows a classifier based on overall fragmentomic signatures of all microbial molecules in each sample is used for determining whether or not a patient is infected and monitoring the antibiotics treatment response.

Plasma cell-free DNA can be subjected to massively parallel sequencing (e.g. Illumina high-throughput sequencing based on sequencing by synthesis technology). The paired-end sequencing reads aligned to the reference human genome (for example, hg38) were first removed to obtain sequenced reads that are not aligned to the human genome. Such sequence reads would be enriched for microbial reads. The microbial species would then be determined by aligning these non-human genome-aligned sequenced reads to a set of microbial reference genomes. From the alignment results, the microbial origin of these sequencing reads could be determined. In some instances, the taxonomic ranks of microbes identified from plasma DNA sequencing could include but not limited to kingdom, phylum, class, order, family, genus and species.

The alignment procedure could be performed using various software packages, such as Kraken2, BLAST, FASTA, Bowtie, BWA, BFAST, SHRiMP, SSAHA2, NovoAlign and SOAP (Wood et al. Genome Biol. 2019; 20:257; Li et al. Bioinformatics. 2009; 25:1754-1760; Langmead et al. Nat Methods. 2012; 9:357-359; McGinnis et al. Nucleic Acids Res. 2004; 32: W20-W25; Lipman et al. Science. 1985; 227:1435-1441; Homer et al. PLoS ONE. 2009; 4: e7767; Rumble et al. PLoS Comput. Biol. 2009; 5: e1000386; Ning et al. Genome Res. 2001; 11:1725-1729; Li et al. Bioinformatics. 2008; 24:713-714). The microbial genome corresponding to the optimal alignment of a sequenced microbial DNA fragment would be identified as the candidate microbial species contributing such a microbial fragment. The optimal alignment could be defined as the highest mapping quality score that indicated the Phred-scaled probability of a read being misplaced (i.e. −10 log E where ‘E’ represents the error probability).

The microbial DNA fragment size could be determined by the number of nucleotides between the outermost genomic coordinates of paired-end sequencing reads of a DNA fragment. In some embodiments, the fragment sizes are used for differentiating microbial DNA from contaminant DNA sequences (FIG. 3A (i)). From the alignment results, the end motifs of microbial DNA fragment can be determined as a number of nucleotides at the ends of paired-end reads. For example, the frequency of each 4-mer end motif (i.e. end signatures) could be calculated from paired-end reads aligned to microbial reference genomes, and are denoted as observed end motif frequency. The frequency of a 4-mer motif present in a microbial reference genome was determined using a sliding window of 4 bases across the microbial reference genome, termed expected end motif frequency (i.e. the proportion of 4-mer motif existing in a microbial reference genome). In some instances, the overall expected end motif frequency could be defined by the expected end motif frequency in a number of microbial reference genomes.

In some embodiments, the ratio of the observed frequency to the expected frequency (i.e., O/E ratio) for an end motif derived from a microbial reference genome is calculated for downstream analysis. In some embodiments, the hierarchical clustering analysis is used for classifying the microbial cell-free DNA and contaminant DNA molecules on the basis of end motifs (FIG. 3A (ii)). In some embodiments, different statistical approaches are used to analyse a number of end signatures, for example but not limited to, logistic regression, support vector machines (SVM), decision tree, naïve Bayes classification, clustering algorithm, principal component analysis, singular value decomposition (SVD), t-distributed stochastic neighbor embedding (tSNE), artificial neural network, and ensemble methods which construct a set of classifiers and then classify new data points by taking a weighted voting of their prediction (FIG. 3A (iii)). In some embodiments, microbial DNA abundance between different human subjects with and without diseases (e.g., infection) is analyzed using those sequenced reads carrying end motifs that were underrepresented in contaminant DNA but overrepresented in microbial DNA (FIG. 3B). In some instances, when the O/E ratio for an end motif exceeded a certain threshold, such an end motif would be considered to be over-represented. In some instances, such threshold could be, but not limited to, 1.1, 1.2, 1.3, 1.4, 1.5, 1.6, 1.7, 1.8, 1.9, 2, 3, 4, 5, 10, 20, 30, etc.

### C. Level of Infection

The level of infection can indicate the presence, absence, or an amount of infective microorganisms in the biological sample. The amount of pathogens can be used to predict the amount, degree, or severity of infections in the subject. For example, procalcitonin (PCT) is a peptide precursor of the hormone calcitonin and can be used to predict a response to bacterial infections. In healthy subjects, the level of PCT in the blood stream is below the limit of detection of clinical assay (Dandona et al. J Clin Endocrinol Metab. 1994; 79:1605-8). Conversely, high levels of PCT can indicate severe infections in the subjects, including sepsis.

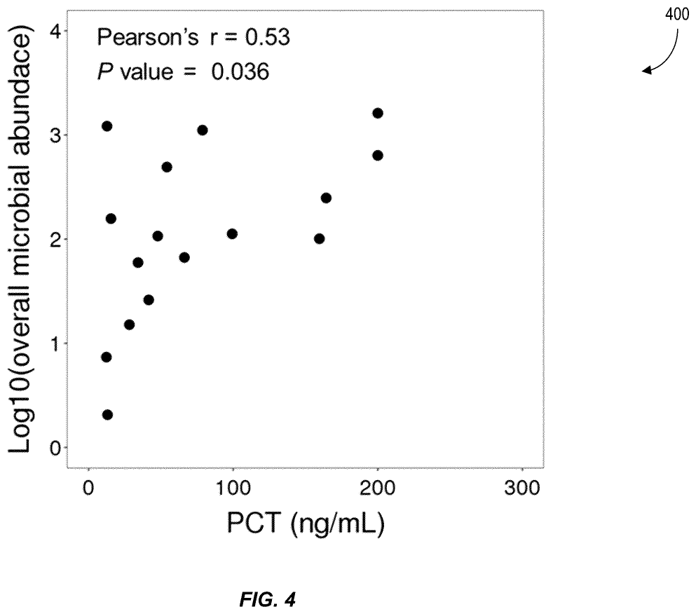

FIG. 4 shows a graph 400 that identifies a correlation between PCT level and overall microbial cell-free DNA abundance, according to some embodiments. As shown in the FIG. 4, a positive correlation between overall microbial cfDNA abundance (reads per million (RPM) in log 10 scale) and PCT level (ng/mL) was identified in subjects with infection. For example, a high number of microbial sequence reads was associated with up to 200 ng/mL of PCT. The graph 400 suggests that the amount of microbial cell-free DNA detected by sequencing could be used to predict the severity of infectious diseases.

### III. Size Analysis of Microbial Cell-Free DNA Fragments

Infection-causing pathogen-derived microbial DNA can be distinguished from contaminant DNA based on their respective sizes. Cell-free DNA molecules being associated from one or more microbe reference genomes (e.g., via sequencing and alignment) can be identified. A size for each of the cell-free DNA molecules can be determined. The sizes of the cell-free DNA molecules of the one or more microbe reference genomes can used to determine a statistical value. In some instances, the statistical value is an average, mode, median, or mean of a size distribution of the cell-free DNA molecules of the one or more microbe reference genomes. The statistical value is compared with a cutoff value for determining a level of infection in the subject.

### A. Size Characteristics of Microbial Cell-Free DNA Fragments

To determine sizes of microbial cell-free DNA fragments, we have sequenced plasma DNA samples from 16 patients with infection who were confirmed to have bacterial or fungal infection via blood culture or body fluid culture. The sites of infection include biliary (6/16), lung (3/16), liver (2/16), abdomen (2/16), bowel (1/16) and urinary tract (1/16). In addition, we also sequenced plasma DNA samples from other 12 patients who have no infection. The sample has a median number of 127 million paired-end reads (range: 53-179 million). Thirty no-template control (NTC) samples were prepared in parallel.

In the following experiment, contaminant DNA was defined as microbial DNA fragments detected from the NTC samples, including Pseudomonas, Variovorax, Acidovorax, etc. In some embodiments, the contaminant DNA is defined as the top 30 microbes (in abundance) detected in the NTC samples. Additionally or alternatively, the list of contaminant microbes can be obtained from public databases or previously reported publications. Although the NTC samples should include contaminant DNA only, the NTC samples may incidentally include microbial DNA of the same species as pathogens. The pathogens were defined as microbes which were confirmed with microbiology test (blood culture, body fluid culture, etc.). For the purposes of this experiment, a clinical sample having the infection-causing pathogen-derived microbial DNA is obtained from a person identified as having pathogens that are known to cause infections. One or more culture tests can be performed to confirm that the clinical sample includes the microbial DNA molecules that are associated with the known pathogens.

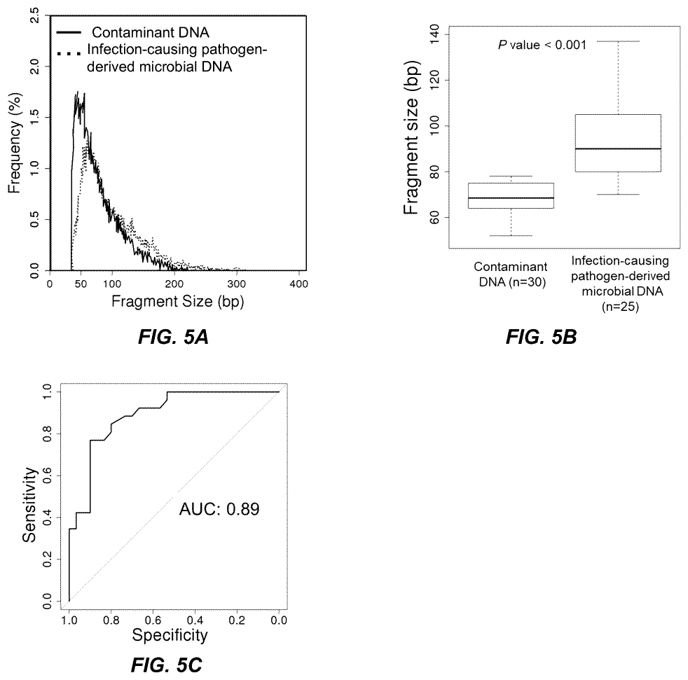

FIGS. 5A-C show a set of diagrams that identify difference in fragment size between infection-causing pathogen-derived microbial DNA and contaminant DNA, according to some embodiments. FIG. 5A shows microbial DNA fragment size distribution between contaminant DNA and infection-causing pathogen-derived microbial DNA. FIG. 5B shows a boxplot of microbial DNA fragment sizes for plasma infection-causing pathogen-derived microbial DNA and contaminant DNA. FIG. 5C shows ROC analysis for distinguishing plasma infection-causing pathogen-derived microbial DNA from contaminant DNA on the basis of the median microbial DNA fragment size.

As shown in FIG. 5A, the fragment size profile of contaminant DNA was shifted towards the left of that of infection-causing pathogen-derived microbial DNA in the plasma of patients with infection. The result suggests that short DNA molecules were enriched with contaminant DNA molecules, while the longer DNA molecules were associated with infection-causing pathogen-derived microbial DNA (FIG. 5A). In FIG. 5B, the contaminant DNA showed a median size of 69 bp. In contrast, the median size of infection-causing pathogen-derived microbial DNA (90 bp) was longer than contaminant DNA (P value: 0.001; Mann-Whitney U test) (FIG. 5B). The results in FIGS. 5A-B suggest that the fragmentation process occurring in the reagents or exposure to other elements appears to cause more fragmentation in contaminant DNA than the fragmentation in the infection-causing pathogen-derived microbial DNA, thereby resulting in shorter fragments in contaminant DNA and longer fragments in infection-causing pathogen-derived microbial DNA.

In FIG. 5C, the area under (AUC) of receiver operating characteristic (ROC) curve for differentiation between contaminant DNA and infection-causing pathogen-derived microbial DNA based on the median fragment size was 0.89 (FIG. 3C). The results shown in FIG. 3C suggest that the size characteristics of microbial cell-free DNA could be reliably used for differentiating between contaminant DNA and infection-causing pathogen-derived microbial DNA.

### B. Methods for Determining a Level of Infection in a Subject Based on Size Characteristics of Microbial Cell-Free DNA

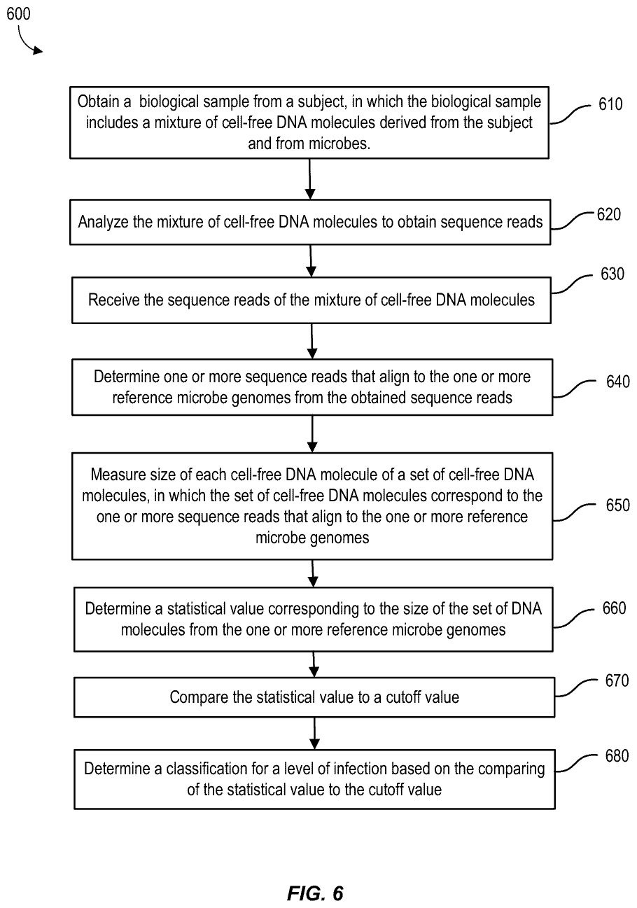

FIG. 6 is a flowchart illustrating a method of determining a level of infection in a biological sample based on size characteristics of microbial cell-free DNA, according to some embodiments. Method 600 can analyze a biological sample to detect infection-causing pathogen-derived microbial cell-free DNA and distinguish over non-pathogenic, contaminant microbial DNA. The detected infection-causing pathogen-derived microbial cell-free DNA can then be used to determine the level of infection (e.g., sepsis). At least a portion of the method may be performed by a computer system.

At block 610, the biological sample is obtained from the subject. As examples, the biological sample can be blood, plasma, serum, urine, saliva, sweat, tears, and sputum, as well as other examples provided herein. In some embodiments (e.g., for blood), the biological sample can be purified for the mixture of cell-free DNA molecules, e.g., centrifuging blood to obtain plasma. The biological sample includes a mixture of cell-free DNA molecules derived from a subject and microbes.

At block 620, the mixture of cell-free DNA molecules of the biological sample are analyzed to obtain sequence reads. The sequencing may be performed in a variety of ways, e.g., using massively parallel sequencing or next-generation sequencing, using single molecule sequencing, and/or using double- or single-stranded DNA sequencing library preparation protocols. The skilled person will appreciate the variety of sequencing techniques that may be used. As part of the sequencing, it is possible that some of the sequence reads may correspond to cellular nucleic acids. A statistically significant number of cell-free DNA molecules can be analyzed so as to provide an accurate determination of the level of infection. In some embodiments, at least 1,000 cell-free DNA molecules are analyzed. In other embodiments, at least 10,000 or 50,000 or 100,000 or 500,000 or 1,000,000 or cell-free DNA molecules, or more, can be analyzed.

The sequencing may be target-capture sequencing as described herein. For example, biological sample can be enriched for DNA molecules from the microbes. The enriching of the biological sample for DNA molecules from the microbes can include using capture probes that bind to a portion of, or an entire genome of, the microbes. The biological sample can be enriched for DNA molecules from a portion of a human genome, e.g., regions of autosomes. In other embodiments, the sequencing include random sequencing.

At block 630, the sequence reads that were obtained from the sequencing of the mixture of cell free DNA molecules are received. The sequence reads may be received by a computer system, which may be communicably coupled to a sequencing device that performed the sequencing, e.g., via wired or wireless communications or via a detachable memory device.

At block 640, one or more sequence reads that align to the one or more reference microbe genomes are determined from the obtained sequence reads. Each of the reference microbe genomes corresponds to a particular species of microbes. The particular microbe species can correspond to a species of one of the microbial genera consisting of Bacteroides, Klebsiella, Escherichia, Enterobacter, Citrobacter, Aeromonas, Mycobacterium, Candida, Prevotella, Streptococcus, and Orientia. In some instances, the aligned sequence reads to a particular microbe species (e.g., B. fragilis) are reclassified as being from a corresponding genus (e.g., Bacteroides). If the aligned sequence reads include infection-causing pathogen-derived microbial DNA, then it can be determined that the infection-causing pathogen-derived microbial DNA are associated with Bacteroides.

In some embodiments, one or more sequence reads that include both ends of the nucleic acid fragment can be received. Thus, a plurality of sequence reads can be obtained from a sequencing of the mixture of cell free nucleic acid molecules. The one or more sequence reads can be aligned to the reference genome to obtain one or more aligned locations. The one or more aligned locations can be used to determine the size of the nucleic acid fragment.

Before the sequencing is performed, one or more assays can be used to determine whether a sufficient amount of the microbes is detected, and therefore warrants the sequencing to be performed. In some implementations, real-time polymerase chain reaction (PCR) can be performed using the biological sample or a different biological sample obtained from the subject contemporaneously (e.g., same clinical visit) as the biological sample. The real-time PCR can provide a quantity of nucleic acid molecules from the microbes using techniques described herein or known to one skilled in the art, e.g., using Ct values. The quantity can be compared to a quantity threshold. When the quantity is above the quantity threshold, the sequencing can be performed, thereby not wasting resources sequencing samples that do not have a sufficient quantity of microbial nucleic acids to warrant the more accurate technique. In some embodiments, digital PCR could be used instead of sequencing. The capture probes can be used with corresponding primers in performing the count of sequence reads.

In some instances, the aligned sequence reads are determined by filtering out sequences that align to a human reference genome. For example, the sequence reads can be aligned to the human reference genome. A subset of sequence reads aligning to the human reference genome can be filtered out. The remaining sequence reads can then be realigned to the one or more reference microbe genomes. A sequence read of the remaining sequence reads that aligns to a reference microbe genome of a particular microbe species (e.g., B. fragilis) can be determined the sequence read as being associated the particular microbe species.

The following blocks 650 to 680 can be iterated for each reference microbe genome of the one or more reference microbe genomes. For example, the level of infection for a particular microbial species can be determined based on analyzing sequence reads that align to the corresponding reference microbe genome. In some instances, the level of infection can be determined across multiple microbial species by iterating blocks 650 to 680 through multiple reference microbe genomes.

Additionally or alternatively, blocks 650 to 680 are performed once for all of the one or more reference microbe genomes, in which the level of infection would be based on microbial species that correspond to the one or more reference microbe genomes.

At block 650, a size of each cell-free DNA molecule of a set of cell-free DNA molecules is measured, in which the set of cell-free DNA molecules correspond to the one or more sequence reads that align to the one or more reference microbe genomes. In other words, each cell-free DNA molecule of a set of cell-free DNA molecules is determined to be from a respective reference microbe genome of the one or more reference microbe genomes. The size may be measured via any suitable method, for example, methods described above. As examples, the measured size can be a length, a molecular mass, or a measured parameter that is proportional to the length.

In some embodiments, both ends of a DNA molecule can be sequenced and aligned to a genome to determine starting and ending coordinates of the DNA molecule, thereby obtaining a length in bases, which is an example of size. Such sequencing can be target-capture sequencing, e.g., involving capture probes as described herein. Other example techniques for determining size include electrophoresis, optical methods, fluorescence-based method, probe-based methods, digital PCR, rolling circle amplification, mass spectrometry, melting analysis (or melting curve analysis), molecular sieving, etc. As an example for mass spectrometry, a longer molecule would have a larger mass (an example of a size value).

At block 660, a statistical value corresponding to the size of the set of DNA molecules from the one or more reference microbe genomes is determined. The statistical value can correspond to a size distribution of the set of DNA molecules from the one or more reference microbe genomes (e.g., a size profile). A cumulative frequency of fragments smaller or larger than a size threshold is an example of a statistical value. The statistical value can provide a measure of the overall size distribution, e.g., an amount of small fragments relative to an amount of large fragments.

In some embodiments, the statistical value can be an average, mode, median, or mean of the size distribution. Additionally or alternatively, the statistical value can be a percentage of the plurality of DNA molecules in the biological sample from the one or more reference microbe genomes that exceed a size threshold (e.g., 50 bp). For such a statistical value, the presence of infection-causing pathogen-derived microbial cell-free DNA molecules can be identified in the mixture of cell-free DNA molecules, when statistical value is above the cutoff value.

At block 670, the statistical value is compared to a cutoff value. It can be determined whether the statistical value exceeds the cutoff value (e.g., above or below, depending on how the statistical value is defined). In some embodiments, the statistical value corresponds to a mean fragment size of the plurality of DNA molecules. The cutoff value for the mean size value can be a numerical value selected between 75-90 bp. If the statistical value is above the selected cutoff value (e.g., 80 bp), then the presence of infection-causing pathogen-derived microbial cell-free DNA molecules can be identified since infection-causing pathogen-derived microbial DNA are associated with longer fragments. In some embodiments, the cutoff values could include but not limited to 40 bp, 50 bp, 60 bp, 70 bp, bp, 100 bp, 110 bp, 120 bp, 130 bp, 140 bp, 150 bp, 160 bp, 170 bp, 180 bp, 190 bp, 200 bp, 250 bp, 300 bp, 350 bp, 400 bp, 500 bp etc.

Additionally or alternatively, the statistical value corresponds to a percentage of the plurality of DNA molecules from the one or more reference microbe genomes that are below a size threshold (e.g., 50 bp). The cutoff value for the percentage value can be a value selected between 1.25-1.75%. If the statistical value is below the selected cutoff value (e.g., 1.5%), then the presence of infection-causing pathogen-derived microbial cell-free DNA molecules can be identified. In some embodiments, the cutoff values could include but not limited to 1.29%, 1.30%, 1.35%, 1.40%, 1.45%, 1.50%, 1.55%, 1.60%, 1.65%, 1.70%, 1.75% etc.

If the statistical value does not exceed the cutoff value, it can be determined that the plurality of cell-free DNA molecules of the reference microbe genomes correspond to contaminant DNA. In some instances, the cutoff value is selected based on microbe species or genera associated with the one or more reference genomes.

At block 680, a classification for a level of infection is determined based on the comparing of the statistical value to the cutoff value. The level of infection can indicate the presence, absence, or an amount of infective microorganisms in the biological sample. In some instances, the level of infection indicates infection at a particular site, in which the infection site originates from biliary, lung, liver, abdomen, bowel, and/or urinary tract. Different cutoff values can be selected to increase the sensitivity and/or specificity for predicting the level of infection. In some embodiments, the cutoff value can be selected such that a sensitivity of determining the classification of level of infection is at least 80% and a specificity of determining the classification of level of infection is at least 70%. Additionally or alternatively, the classification can also identify that the biological sample includes infection-causing pathogen-derived microbial DNA and further provides a particular genus or species of microbes (e.g., Bacteroides) that are associated with the infection-causing pathogen-derived microbial DNA.

In some instances, the cutoff value can be adjusted to increase specificity while compensating for slight decrease in sensitivity, or vice versa. In effect, the selection of the cutoff values can identify an optimal sensitivity and specificity for determining the level of infection of the subject.

### IV. End Signature Analysis of Microbial Cell-Free DNA

Infection-causing pathogen-derived microbial DNA can be distinguished from contaminant DNA based on sequence end signatures. In some instances, one or more specific end signatures are used (e.g., CC, GG) to determine presence of infection-causing pathogen-derived microbial DNA in a biological sample. In particular, an amount of sequence reads having ending sequences that correspond to one or more sequence end signatures can be determined. The amount can be used to identify a parameter for determining a level of infection in the subject. The parameter can indicate a presence of infection-causing pathogen-derived microbial DNA in the subject. For example, the parameter can be a frequency of sequences exhibiting the one or more end signatures. In some instances, the parameter is determined based on a ratio between an observed frequency of sequences exhibiting the one or more end signatures versus the expected frequency of sequences exhibiting the one or more end signatures.

Additionally or alternatively, the parameter can be a ratio of a first observed frequency for a first sequence end signature and a second observed frequency for a second sequence end signature. In some instances, the parameter can be a ratio value between a first ratio for a first sequence end signature and a second O/E ratio for a second sequence end signature.

### A. 1-mer End Signatures of Infection-Causing Pathogen-Derived Microbial Cell-Free DNA

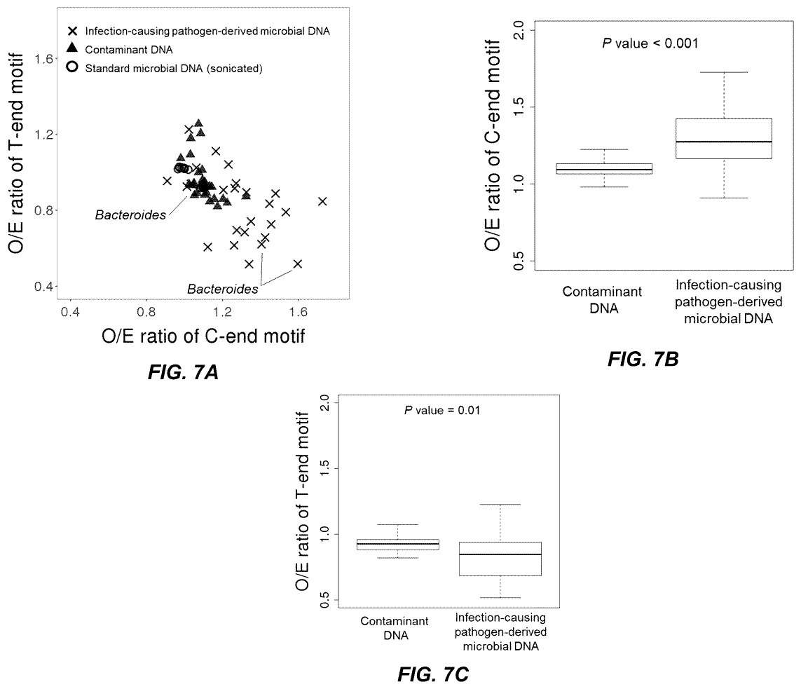

We further studied the end motif profiles of microbial DNA fragments. In some embodiments, one could use 1-mer end motif, namely A-end, C-end, G-end, and T-end. FIGS. 7A-C show diagrams that identify different preferences in 1-mer end signatures for infection-causing pathogen-derived microbial DNA and contaminant DNA, according to some embodiments. FIG. 7A shows a scatter plot showing the differences in C-end and T-end preference of contaminant DNA molecules and infection-causing pathogen-derived microbial DNA molecules. In FIG. 7A, the circles represent sonicated microbial genomic DNA. The triangles represent contaminant microbial DNA detected in NTC samples. The crosses represent infection-causing pathogen-derived microbial DNA detected in plasma. The x-axis of FIG. 7A corresponds to O/E ratios for fragments with C-end motif, and the y-axis of FIG. 7A corresponds to O/E ratios for fragments with T-end motif.

FIG. 7B shows a boxplot comparing the O/E ratio of C-end motif between contaminant DNA and infection-causing pathogen-derived microbial DNA. FIG. 7C shows a boxplot comparing the O/E ratio of T-end motif between contaminant DNA and infection-causing pathogen-derived microbial DNA.

In FIGS. 7A-C, O/E ratio of C-end and T-end motifs were compared between contaminant DNA in NTC samples and infection-causing pathogen-derived microbial DNA in plasma samples of patients with infection. In addition, we have included reference samples with microbial genomic DNA treated with sonication. The reference sample provides additional data points for identifying sequence end signatures associated with the infection-causing pathogen-derived microbial DNA. The sonication of the microbial DNA in the reference sample is performed to create random fragments of the microbial DNA molecules. The random fragments in the reference sample can provide a similar behavior relative to the fragments of contaminant DNA, in which end motifs in both groups of fragments can be used to identify sequence end signatures of the infection-causing pathogen-derived microbial DNA.

Additionally, a clinical sample having the infection-causing pathogen-derived microbial DNA is obtained from a person identified as having pathogens that are known to cause infections. One or more cell culture tests can be performed to confirm that the clinical sample includes the microbial DNA molecules that are associated with the known pathogens.

As shown in FIG. 7A, contaminant microbial DNA from NTC samples had O/E ratios of both C-end and T-end motif close to 1, which were similar to the reference samples with microbial DNA prepared by sonication. In contrast, for infection-causing pathogen-derived microbial DNA fragments present in patients with infection, the O/E ratios of fragments with T-end and C-end motif were located in a different cluster from those of the contaminant microbial DNA and reference microbial DNA. As shown in FIG. 7B, C-end fragments were enriched in infection-causing pathogen-derived microbial DNA (median O/E ratio of C-end motif: 1.27), in comparison with contaminant DNA identified in NTC samples (median O/E ratio of C-end motif: 1.09). FIG. 7C shows different ending-sequence characteristics between contaminant DNA and infection-causing pathogen-derived microbial DNA. In particular, T-end fragments were under-represented in infection-causing pathogen-derived microbial DNA (median O/E ratio of T-end motif: 0.85), compared with contaminant DNA identified from NTC samples (median O/E ratio of T-end motif: 0.93).

These data suggested that microbial DNA fragments derived from contaminant microbes and pathogenic microbes would contain different end motifs. As shown in FIG. 7A, the two end signatures (C-end and T-end motif) can both be utilized to determine whether a microorganism is pathogenic or not by analysing the corresponding microbial DNA in plasma. As an illustrative example, we have analysed two patients with their body fluid culture positive for Bacteroides. At the same time, the bacteria Bacteroides was also found in the NTC samples of the same sequencing run. An approach would classify Bacteroides as a contaminant microbe because of its presence in NTC samples. The end signature analysis showed that end signatures of Bacteroides-derived DNA found in plasma of this patient were different from that of Bacteroides-derived DNA in the NTC samples (FIG. 7A), but similar to the other infection-causing pathogen-derived microbial DNA. These results suggested that the end signature analysis would improve the accuracy of data interpretation of microbial DNA fragments in plasma.

### B. 2-mer End Signatures of Infection-Causing Pathogen-Derived Microbial Cell-Free DNA

In some embodiments, 2-mer end motifs, including CC-end and GG-end, are used to differentiate contaminant DNA from infection-causing pathogen-derived microbial DNA. We first compare O/E ratios of fragments with 2-mer end motifs between contaminant DNA and infection-causing pathogen-derived microbial DNA. Reference samples with microbial genomic DNA treated with sonication were also included. The reference sample provides additional data points for identifying sequence end signatures associated with the infection-causing pathogen-derived microbial DNA. The sonication of the microbial DNA in the reference sample is performed to create random fragments of the microbial DNA molecules. The random fragments in the reference sample can provide a similar behavior relative to the fragments of contaminant DNA.

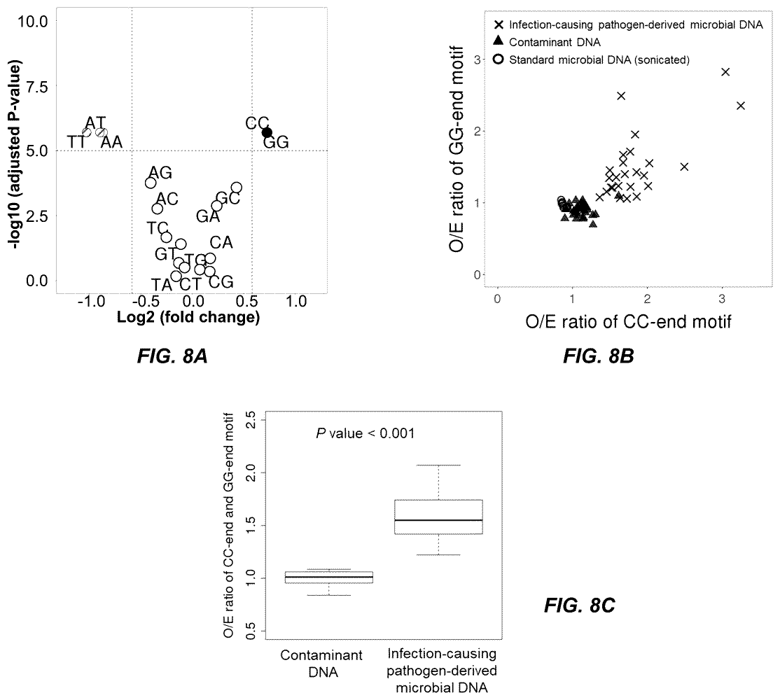

FIGS. 8A-C show diagrams that identify different preference in 2-mer end signatures for infection-causing pathogen-derived microbial DNA and contaminant DNA, according to some embodiments. FIG. 8A shows a volcano plot showing the fold-change versus statistical significance (FDR adjusted P value) comparing infection-causing pathogen-derived microbial cell-free DNA and contaminant DNA in terms of O/E ratios of 2-mer end motifs. The significantly overrepresented (fold change>1.5, −log 10 P>5) and underrepresented (fold change<0.67, −log 10 P>5) end motifs in infection-causing pathogen-derived microbial cell-free DNA were denoted in red and blue color respectively. FIG. 8B shows a scatter plot showing the difference in CC-end and GG-end preference. The circles represent sonicated microbial genomic DNA, the triangles represent contaminant microbial DNA detected in NTC samples, and the crosses represent infection-causing pathogen-derived microbial DNA detected in plasma. FIG. 8C shows a boxplot comparing the O/E ratios of CC-end and GG-end motifs between contaminant DNA and infection-causing pathogen-derived microbial DNA.

As shown in FIG. 8A, the clustering analysis showed that CC-end and GG-end were the mostly overrepresented 2-mer end motifs in infection-causing pathogen-derived microbial DNA (Mann-Whitney U test). The overrepresentation of CC-ends and GG-ends can be attributed to the preferential cutting of microbial cell-free DNA molecules by nucleases while they are present in the plasma sample. In addition, the clustering analysis showed underrepresentation of AA-end, AT-end, and TT-end. The underrepresentation of certain end motifs can also be attributed to nuclease activity. By contrast, the contaminant DNA did not show specific overrepresentation or underrepresentation of end motifs because they are introduced (with reagents) to the sample after nucleases are removed from the biological sample. Due to the lack of nuclease activity, the contaminant DNA shows random pattern of end motifs.

In FIG. 8B, O/E ratios of fragments with CC-end motif were plotted against O/E ratios of fragments with GG-end motif for sonicated microbial DNA, contaminant DNA, and infection-causing pathogen-derived microbial DNA, respectively. Contaminant microbial DNA had O/E ratios of both CC-end motif and GG-end motif close to 1, which was similar to the microbial DNA prepared by sonication. In contrast, the infection-causing pathogen-derived microbial DNA fragments present in patients with infection were located in a different cluster from the contaminant microbial DNA according to the O/E ratios of CC-end motif and GG-end motif. Further, the infection-causing pathogen-derived microbial DNA showed a ratio value that is greater than 1, in which the ratio value is determined between the O/E ratios of fragments with CC-end motif and GG-end motif.

In some embodiments, the two end signatures (O/E ratios of CC-end motif and GG-end motif) are utilized separately to determine whether a microorganism is pathogenic or not by analysing the corresponding microbial DNA in plasma. As shown above, the two end signatures can be compared against each other to identify infection-causing pathogen-derived microbial DNA. Additionally or alternatively, FIG. 8C shows that a combined signature, named O/E ratio of CC-end and GG-end motif, can be effectively used to determine whether a microorganism is actually present in a testing sample but not introduced during experiment procedures. In particular, the separation between contaminant DNA and infection-causing pathogen-derived microbial DNA is substantially greater.

### C. Identifying Patients with Infection Using End Signatures of Pathogenic Microbial Cell-Free DNA

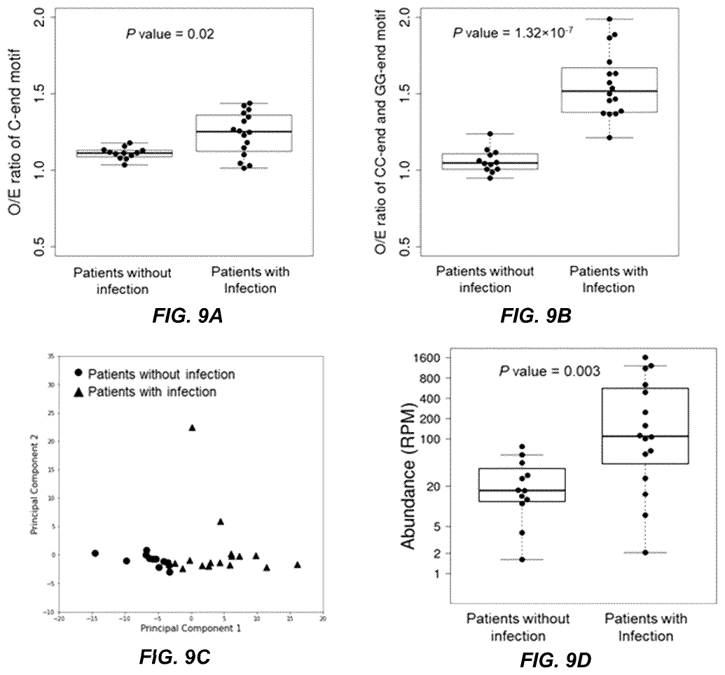

We further compared the overall microbial DNA end signatures between the patients with infection and patients without infection. The overall microbial DNA was defined as all of the microbial sequences including contaminant microbial DNA and infection-causing pathogen-derived microbial DNA (if it exists) determined in one sample. FIGS. 9A-D show diagrams that identify a comparison of cases with and without infection regarding the overall end signatures of microbial DNA, according to some embodiments. FIG. 9A shows a boxplot showing the O/E ratio of C-end motif of overall microbial DNA in patients with infection and patients without infection. FIG. 9B shows a boxplot showing the ratio of CC-end and GG-end motif of overall microbial DNA in patients with infection and patients without infection. FIG. 9C shows a scatter plot shows that microbial DNA from patients with infection and patients without infection are clustered into two groups based on principal component analysis (PCA) using O/E ratios of 256 4-mer end motifs of microbial DNA molecules detected in plasma. FIG. 9D shows a boxplot shows the comparison of the microbial DNA abundance between the two groups of patients.

FIG. 9A shows that microbial DNA in patients with infection has a higher preference of C-end compared with that in patients without infection (P value: 0.02). FIG. 9B shows the relative CC-end and GG-end frequency of microbial DNA was higher in the infection group than in the non-infection group (P value: 1.32×10⁻⁷). The results in FIG. 9A demonstrate that the 1-mer end motif can be used for differentiating microbial DNA from infection and non-infection group. As shown in FIGS. 9B and 9D, the performance for differentiating the infection and non-infection group using the two end signatures was much better than using overall microbial DNA abundance (RPM, Reads Per Million sequencing reads) (P value: 0.003). FIG. 9C shows using O/E ratios of 256 4-mer end motifs originating from microbial DNA molecules for both patients with infection or without infection. As shown in FIG. 9C, the two groups of patients tended to be clustered together based on the principal component analysis. The results of FIG. 9C suggest that the overall end signature analysis could be used to determine whether a human subject was infected by pathogens. With respect to FIG. 9D, the comparison of microbial abundance is a different technique used to identify infection-causing pathogen-derived microbial DNA. The lack of precision shown in FIG. 9D demonstrates that the end-signature analyses of FIG. 9B perform better in predicting presence of infection across various biological samples.

### D. Methods for Determining a Level of Infection in a Subject Based on End Signatures of Microbial Cell Free DNA

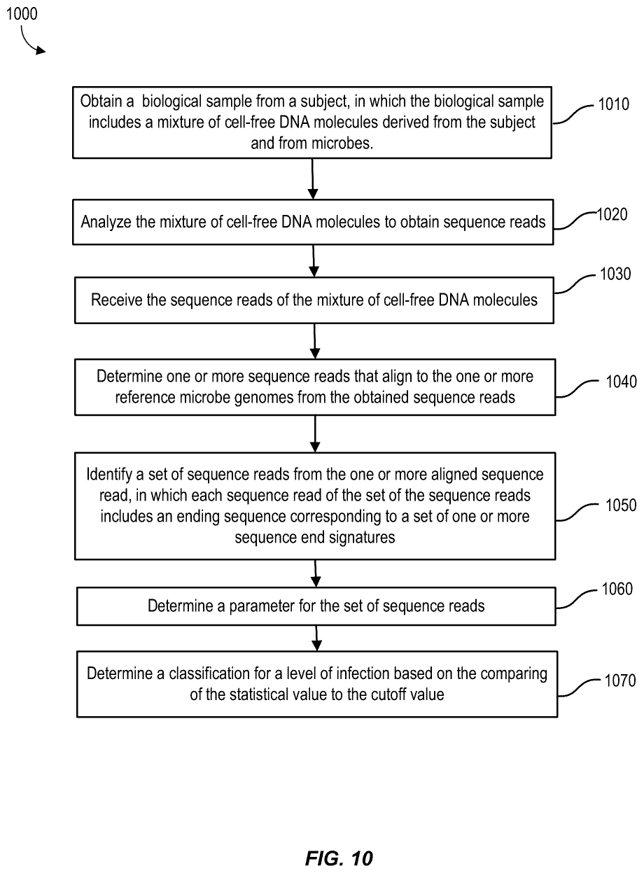

FIG. 10 is a flowchart illustrating a method for determining a level of infection in a biological sample based on sequence end signatures, according to some embodiments. In some instances, the biological sample includes cell-free DNA molecules. Method 1000 can analyze a biological sample to detect infection-causing pathogen-derived microbial cell-free DNA and distinguish over non-pathogenic, contaminant microbial DNA. The detected infection-causing pathogen-derived microbial cell-free DNA can then be used to determine the level of infection (e.g., sepsis). At least a portion of the method may be performed by a computer system.

At block 1010, the biological sample is obtained from the subject. As examples, the biological sample can be blood, plasma, serum, urine, saliva, sweat, tears, and sputum, as well as other examples provided herein. In some embodiments (e.g., for blood), the biological sample can be purified for the mixture of cell-free DNA molecules, e.g., centrifuging blood to obtain plasma. The biological sample includes a mixture of cell-free DNA molecules derived from a subject and microbes.

At block 1020, the mixture of cell-free DNA molecules of the biological sample is analyzed to obtain sequence reads. The sequencing may be performed in a variety of ways, e.g., using massively parallel sequencing or next-generation sequencing, using single molecule sequencing, and/or using double- or single-stranded DNA sequencing library preparation protocols. In some instances, the biological sample is enriched for DNA molecules from the microbes using capture probes that bind to a portion of, or an entire genome of, the microbes. Block 1020 may be performed in a similar manner as block 620 of FIG. 6.

At block 1030, the sequence reads that were obtained from the sequencing of the mixture of cell free DNA molecules are received. The sequence reads include ending sequences corresponding to ends of the cell-free DNA molecules. Block 1030 may be performed in a similar manner as block 630 of FIG. 6.

At block 1040, one or more sequence reads that align to the one or more reference microbe genomes are determined from the obtained sequence reads. Each of the reference microbe genomes corresponds to a particular species of microbes. The particular microbe species can correspond to a species of one of the microbial genera consisting of Bacteroides, Klebsiella, Escherichia, Enterobacter, Citrobacter, Aeromonas, Mycobacterium, Candida, Prevotella, Streptococcus, and Orientia. Block 1040 may be performed in a similar manner as block 640 of FIG. 6.

In some embodiments, one or more sequence reads that include both ends of the nucleic acid fragment can be received. Thus, a plurality of sequence reads can be obtained from a sequencing of the mixture of cell free nucleic acid molecules. The one or more sequence reads can be aligned to the reference genome to obtain one or more aligned locations. The one or more aligned locations can be used to determine the size of the nucleic acid fragment.

Before the sequencing is performed, one or more assays can be used to determine whether a sufficient amount of the microbes is detected, and therefore warrants the sequencing to be performed. In some implementations, real-time polymerase chain reaction (PCR) can be performed using the biological sample or a different biological sample obtained from the subject contemporaneously (e.g., same clinical visit) as the biological sample. The real-time PCR can provide a quantity of nucleic acid molecules from the microbes using techniques described herein or known to one skilled in the art, e.g., using Ct values. The quantity can be compared to a quantity threshold. When the quantity is above the quantity threshold, the sequencing can be performed, thereby not wasting resources sequencing samples that do not have a sufficient quantity of microbial nucleic acids to warrant the more accurate technique. In some embodiments, digital PCR could be used instead of sequencing. The capture probes can be used with corresponding primers in performing the count of sequence reads.

In some instances, the aligned sequence reads are determined by filtering out sequences that align to a human reference genome. For example, the sequence reads can be aligned to the human reference genome. A subset of sequence reads aligning to the human reference genome can be filtered out. The remaining, non-aligned sequence reads can then be realigned to the one or more reference microbe genomes. A sequence read of the remaining sequence reads that aligns to a reference microbe genome of a particular microbe species (e.g., B. fragilis) can be determined the sequence read as being associated the particular microbe species.

The following blocks 1050 to 1070 can be iterated for each reference microbe genome of the one or more reference microbe genomes. For example, the level of infection for a particular microbial species can be determined based on analyzing sequence reads that align to the a corresponding reference microbe genome. In some instances, the level of infection can be determined across multiple microbial species by iterating blocks 1050 to 1070 through multiple reference microbe genomes.

In some instances, blocks 1050 to 1070 are performed once for all of the one or more reference microbe genomes, in which the level of infection would be based on microbial species that correspond to the one or more reference microbe genomes.

At block 1050, a set of sequence reads are identified from the one or more aligned sequence reads. In some embodiments, each sequence read of the set of the sequence reads includes an ending sequence corresponding to a set of one or more sequence end signatures. The ending sequences may be determined using the one or more reference microbe genomes, e.g., to identify bases just before a start position or just after an end position. Such bases will still correspond to ends of cell-free DNA fragments, e.g., as they are identified based on the ending sequences of the fragments.

The set of sequence reads can correspond to a single end signature of the set of end signatures. Additionally or alternatively, the set of sequence reads can correspond to two or more end signatures of the set of sequence end signatures. For example, a first sequence read of the set of sequence reads can include an ending sequence that corresponds to a first sequence end signature, and a second sequence read of the set of sequence reads can include an ending sequence that corresponds to a second sequence end signature.

In some instances, a sequence end signature of the set of one or more sequence end signatures corresponds to a single nucleotide (e.g., 1-mer) end motif, such as A-end, C-end, G-end, and T-end. For example, if there are two sequence end signatures, a first sequence read of the set of sequence reads can include an ending sequence that corresponds to a C-end signature, and a second sequence read of the set of sequence reads can include another ending sequence that corresponds to a T-end signature.

In some instances, a sequence end signature of the set of one or more sequence end signatures corresponds to a 2-mer end motif, such as CC-end, GG-end, AA-end, TT-end, and AT-end. For example, if there are two sequence end signatures, a first sequence read of the set of sequence reads can include an ending sequence that corresponds to a CC-end signature, and a second sequence read of the set of sequence reads can include another ending sequence that corresponds to a GG-end signature.

Additionally or alternatively, the set of one or more sequence end signatures can include end motifs having various lengths. For example, a first sequence end signature can correspond to a 1-mer end motif, a second and third sequence end signatures can correspond to 2-mer end motif. The skilled person will appreciate different types of sequence end signatures that can be used to identify the set of sequence reads (e.g., 3-mer, 4-mer, 6-mer).

At block 1060, a parameter of the set of the sequence reads is determined. The parameter can be utilized to determine whether a microorganism associated with the set of sequence reads is pathogenic. In some embodiments, the parameter corresponds to a first amount (e.g., a count) of the set of the sequence reads. The parameter can be a frequency derived based on the count of the set of the sequence reads over a total count of the aligned sequence reads.

In some instances, the parameter is a ratio value (e.g., an O/E ratio) derived from between the first frequency of the set of sequence reads (e.g., sequence reads having T-ends, sequence reads having GG-ends) and a corresponding expected frequency. The expected frequency can correspond to frequency of sequence reads for a reference sample, in which the sequence reads of the reference sample include ending sequences corresponding to a sequence end signature of the set of one or more sequence end signatures.

In some embodiments, the parameter is a combined value of a first O/E ratio for a first sequence end signature and a second O/E ratio for a second sequence end signature. In some instances, the parameter is a ratio between the first O/E ratio for the first sequence end signature and the second O/E ratio for the second sequence end signature. The first O/E ratio can be derived based on: (i) an observed frequency of a first subset of sequence reads that correspond to the first sequence end signature (CC-end) of the set of sequence end signatures; and (ii) a corresponding expected frequency for the first sequence end signature. The second ratio can be derived based on: (i) a frequency of a second subset of sequence reads that correspond to the second sequence end signature (GG-end) of the set of sequence end signatures; and (ii) a corresponding expected frequency for the second sequence end signature.

Additionally or alternatively, the parameter can be determined by analyzing a plurality of O/E ratios for the set of sequence reads, in which each ratio of the plurality of ratio can be derived based on: (i) a frequency of a subset of sequence reads that correspond to a respective sequence end signature of the set of sequence end signatures; and (ii) a corresponding expected frequency for the respective sequence end signature.

At block 1070, a classification for a level of infection is determined based on the parameter. The level of infection can indicate the presence, absence, or an amount of infective microorganisms in the biological sample. In some instances, the level of infection indicates infection at a particular site, in which the infection site originates from biliary, lung, liver, abdomen, bowel, and/or urinary tract. Different reference values can be selected to increase the sensitivity and/or specificity for predicting the level of infection. In some embodiments, the reference value can be selected such that a sensitivity of determining the classification of level of infection is at least 80% and a specificity of determining the classification of level of infection is at least 70%. Additionally or alternatively, the classification can also identify that the biological sample includes infection-causing pathogen-derived microbial DNA and further provides one or more genera or species of microbes (e.g., Bacteroides) that are associated with the infection-causing pathogen-derived microbial DNA.

The determination of classification can include comparing the parameter to a reference value. The level of infection can be determined based on whether the parameter exceeds the reference value (e.g., above or below, depending on how the end signatures are defined). Exceeding the reference value can indicate that the mixture of cell-free DNA molecules includes infection-causing pathogen-derived microbial cell-free DNA, which could contribute to a presence of infection in the subject. Conversely, not exceeding the reference value can indicate that the detected microbial cell-free DNA molecules correspond to contaminant DNA.

In some instances, the reference value is selected based on type of sequence end signatures being used for determining the classification for the level of infection. For example, a first reference value for the set of sequence reads having C-end sequences corresponds to a ratio value selected between 1-1.25. If the parameter for the C-end sequences is above the first reference value (e.g., 1.1), then the classification of a presence of infection can be determined. In some embodiments, the first reference value includes but are not limited to 0.9, 1, 1.1, 1.2, 1.3, 1.4, 1.5 etc. In another example, a second reference value for the set of sequence reads having T-end sequences corresponds to a ratio value selected between 0.9-1. If the parameter for the T-end sequences is below the second reference value (e.g., 1), then the classification of a presence of infection can be determined. In some embodiments, the second reference value includes but are not limited to 0.6, 0.7, 0.8, 0.9, 1, 1.1, 1.2 etc. Additionally or alternatively, two or more reference values can be used together to determine the classification for the level of infection.

In some examples for implementing blocks 1060 and 1070, the parameter can be input to a machine learning model (e.g., as described herein). The machine learning model can provide an output classification based on the parameter. A training set can be developed from samples having infection-causing pathogen-derived microbial cell-free DNA. The training of the machine learning model can provide the reference value as well as the formulation for how the reference value is determined. The machine-learning model includes one of logistic regression, support vector machines (SVM), decision tree, naïve Bayes classification, clustering algorithm, principal component analysis, singular value decomposition (SVD), t-distributed stochastic neighbor embedding (tSNE), artificial neural network, or ensemble methods. Process 1000 can terminate thereafter.

### V. Additional Examples for Detecting Infection-Causing Pathogen-Derived Microbial Cell-Free DNA End Signatures

The end-signature analysis can distinguish infection-causing pathogen-derived microbial DNA from contaminant DNA from plasma samples. As shown below, the end-signature analysis of microbial DNA can detect infection-causing pathogen-derived microbial DNA and predict a level of infection across a large number of samples. The results across a public dataset support that the end signatures of microbial cell-free DNA molecules could be used for classifying contaminant and infection-causing pathogen-derived microbial DNA molecules. In addition, the end-signature analysis of microbial DNA can be used across various types of pathogens. For example, the detection of infection-causing pathogen-derived microbial DNA originating from Pseudomonas facilitates accurate classification of level of infection. Such detection of infection-causing pathogen-derived microbial DNA is different other techniques (e.g., NTC samples) that consider DNA molecules from Pseudomonas as contaminant DNA.

### A. Microbial Cell-Free DNA End Signature Analysis in a Large-Scale Sample Cohort from Public Dataset

We analyzed a large sample cohort from a public dataset involving 209 plasma samples from septic subjects. The patients met the sepsis alert clinical criteria, and the samples were confirmed positive by microbiology test and sequencing. Besides, another 170 plasma samples were from asymptomatic subjects (non-septic) (Blauwkamp et al. Nat Microbiol. 2019; 4:663-674). As NTC samples were not included in the published study, the sequences of microbes found in samples of non-septic subjects were referred to as contaminant DNA. The sequences of microbes detected in septic cases that were confirmed with microbiology test and sequencing would be referred to as infection-causing pathogen-derived microbial DNA.

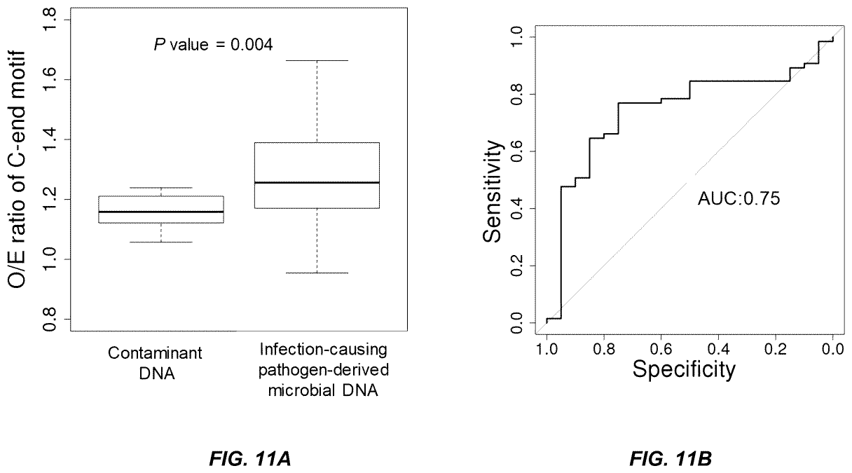

FIGS. 11A-B show diagrams that identify comparisons of the preference regarding end motifs of microbial cell-free DNA and contaminant microbial DNA in a public dataset, according to some embodiments. FIG. 11A shows a boxplot that identifies an O/E ratio of C-end motif of microbial DNA in non-septic cases (i.e., contaminant microbes), infection-causing pathogen-derived microbial DNA detected in septic cases. FIG. 11B shows an ROC curve for distinguishing infection-causing pathogen-derived microbial DNA from contaminant DNA on the basis of the O/E ratio of C-end motif.

As shown in FIGS. 11A-B, we compared the fragment end signatures of infection-causing pathogen-derived microbial DNA from septic patients (in total 65 microbial genus, No. of microbial reads >400) with that of contaminant DNA (in total 20 microbial genus, No. of microbial reads >400) in non-septic samples. In FIG. 11A, the results showed that infection-causing pathogen-derived microbial cell-free DNA had a higher preference for C-ends (P value<0.004). In FIG. 11B, the AUC of the ROC curve concerning the O/E ratio of C-end motif between the contaminant DNA and infection-causing pathogen-derived microbial DNA was 0.75. These results further validated that the end signatures of microbial cell-free DNA molecules could be used for classifying contaminant and infection-causing pathogen-derived microbial DNA molecules in plasma sequencing.

### B. Detecting Infection-Causing Pathogen-Derived Microbial Cell-Free DNA Across Different Types of Microbes

Pseudomonas species are commonly found in the environment, for example in soil and water. It is also an important source of contamination in laboratory. In some instances, P. aeruginosa, for example, could cause pathogenic human infections in the blood, lungs (pneumonia), or other parts of the body. As Pseudomonas is frequently found in NTC samples at a relatively high abundance, it is difficult to differentiate the pathogenic Pseudomonas from contaminant Pseudomonas derived from the environment.

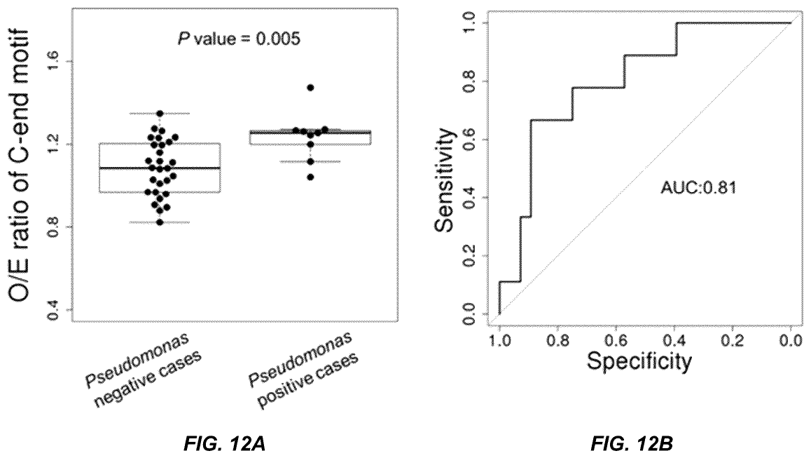

FIGS. 12A-B show diagrams that identify differences in end signatures of contaminant Pseudomonas-derived DNA and pathogenic Pseudomonas-derived DNA, according to some embodiments. Sequence reads corresponding to the samples of Pseudomonas-derived DNA were obtained from a public dataset. For each sample, an amount of sequence reads having a C-end motif was determined, and an O/E ratio of C-end motif was determined based on the amount. The O/E ratios for microbial DNA from samples containing contaminant Pseudomonas-derived DNA were compared from the O/E ratios for samples containing pathogenic Pseudomonas-derived DNA.

FIG. 12A shows a boxplot that identifies a comparison of O/E ratio of C-end motif between Pseudomonas-negative and positive cases. FIG. 12B shows an ROC curve that identifies the performance of end signatures in differentiating Pseudomonas-positive cases from negative cases.

As shown in the boxplot of FIG. 12A, Pseudomonas-derived cell-free DNA fragments detected in plasma of Pseudomonas-positive cases (confirmed with Pseudomonas infection using microbiology test) had higher preference for C-ends, compared with those detected in plasma of Pseudomonas-negative (without Pseudomonas infection using microbiology test) cases (P value: 0.005; Mann-Whitney U test). The ROC curve of FIG. 12B showed that end signature has a good performance in distinguishing truly pathogenic Pseudomonas from contaminant Pseudomonas with an AUC value of 0.81.

### VI. Machine-Learning Techniques for Detecting Infection-Causing Pathogen-Derived Microbial Cell-Free DNA

As the data showed that the end signatures were different between microbial DNA fragments originating from pathogenic microbes and contaminant microbes, end signatures of microbial sequences can be used to determine whether a human subject was exposed to pathogens. In some instances, a plurality of sequence end signatures are used to identify infection-causing pathogen-derived microbial DNA in a biological sample, rather than using one or more specific sequence end signatures. A machine-learning model (e.g., a support vector machine) can process a plurality of vectors to determine whether the biological sample includes infection-causing pathogen-derived microbial DNA from one or more microbe species, in which each vector of the plurality of vectors represents a respective sequence end signature (e.g., CCCA).

### A. Observed and Expected Frequency Ratios of Sequence End Signatures

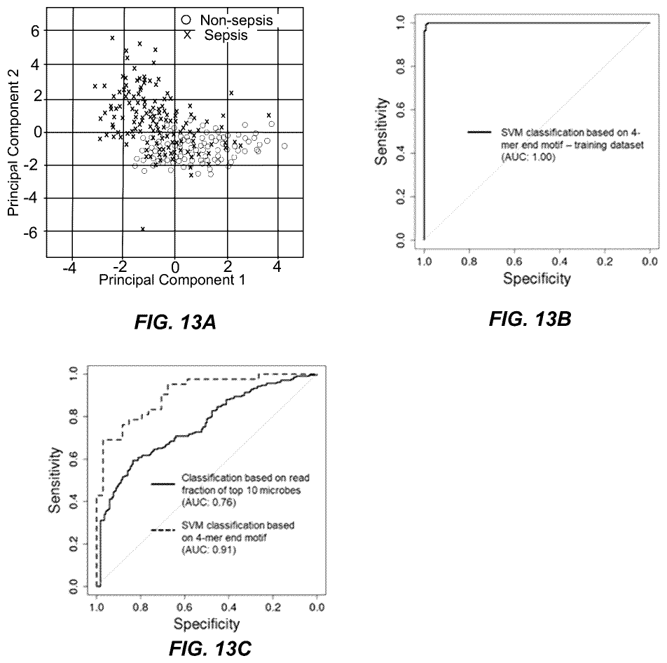

FIGS. 13A-C show diagrams identifying 4-mer end motif signatures that can be applied to distinguish septic cases from non-septic cases, according to some embodiments. Sequence reads corresponding to the samples of septic cases and non-septic cases were obtained from a public dataset. For each sample, an amount of sequence reads corresponding to each of 256 4-mer ends were determined. The amounts of sequence reads for the sample were used to determine the O/E ratios for the sample, at which the O/E ratios were processed using a machine-learning model. The output of the machine-learning model was used to classify whether the sample corresponds to a septic case. The outputs for the samples were compared to evaluate how the machine-learning model performed in distinguishing septic cases from non-septic cases.

FIG. 13A shows a scatter plot shows that septic patients (crosses) and non-septic patients (circles) are clustered into two groups based on principal component analysis (PCA) using 256 4-mer ends of microbial DNA molecules detected in plasma. FIG. 13B shows an ROC curve that identifies performance of SVM classification for distinguishing septic patients from non-septic patients on the basis of O/E ratios of 4-mer end motifs (training dataset). FIG. 13C shows ROC curves that identify performance of SVM classification for distinguishing septic patients from non-septic patients on the basis of O/E ratios of 4-mer end motifs (testing dataset).

FIG. 13A shows that, using the O/E ratios of all the 256 4-mer end motifs from microbial DNA molecules as features, non-septic cases tended to be clustered into different groups from septic cases by principal component analysis. This result also suggested that the 4-mer end motif analysis can be used for classifying the subjects with and without pathogens. In some embodiments, support vector machine (SVM) is used to build a classifier on the basis of 256 end motifs of microbial DNA molecules.

For evaluating the classification performance by the machine-learning model, the dataset was divided into the training dataset (80% samples) and testing dataset (20% samples). The training dataset was used to train the SVM-based classifier with the use of 256 4-mer end motifs as input features. The output value by SVM-based classifier was a probability of being positive for pathogens, ranging from 0 to 1. The higher probability score indicated that one patient would be at higher risk of being infected by pathogens.

FIG. 13B shows that the classification performance of SVM model in the training dataset achieved an AUC value of 1.00. FIG. 13C further shows that accurate classification was achieved for using machine-learning model to predict the level of infection across various samples, with an AUC value of 0.91 in an independent testing dataset. The performance was better than the results generated by abundance-based filtering method (AUC: 0.76). The abundance-based method used the read fraction of top 10 microbes among all the detected microbes found in one sample as a score, with a higher value denoting a higher possibility of infection. The top 10 microbes were used for normalizing the results, as the public dataset did not include any data for background samples.

These results demonstrated that the processing end motifs of microbial fragments using machine-learning model would accurately determine whether a human subject would be infected by microbes. And, such accuracy can be determined without using microbial DNA information from NTC samples. Although SVM model was used for the examples in FIGS. 9A-C, other types of machine-learning models can be used. In some embodiments, machine learning models include but not limited to, linear regression, logistic regression, deep recurrent neural network, Bayes classifier, hidden Markov model (HMM), linear discriminant analysis (LDA), k-means clustering, density-based spatial clustering of applications with noise (DBSCAN), and random forest algorithm.

### B. Methods for Using Machine-Learning Techniques to Determine a Level of Infection in a Subject

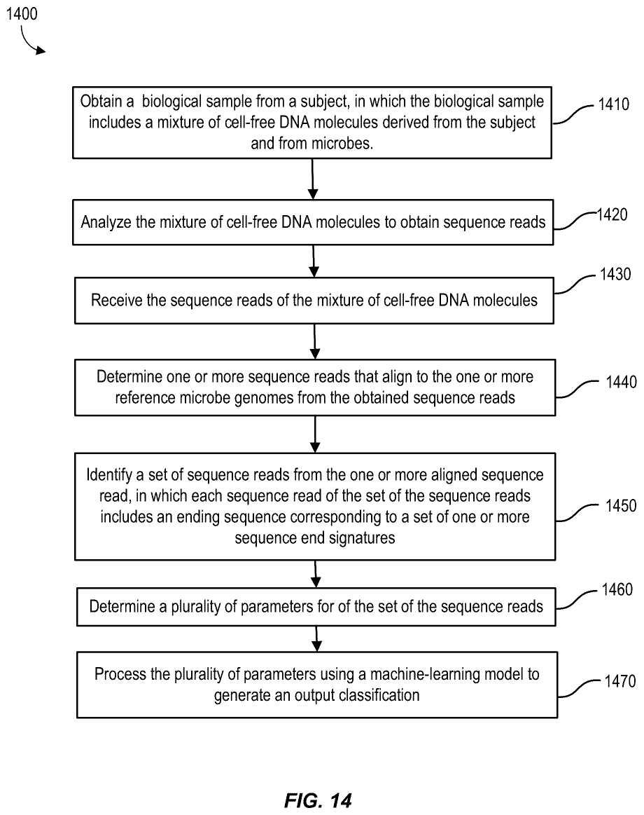

FIG. 14 is a flowchart illustrating a method for using machine-learning techniques to determine a level of infection in a biological sample, according to some embodiments. In some instances, the biological sample includes cell-free DNA molecules. Method 1400 can analyze a biological sample to detect infection-causing pathogen-derived microbial cell-free DNA and distinguish over non-pathogenic, contaminant microbial DNA. The detected infection-causing pathogen-derived microbial cell-free DNA can then be used to determine the level of infection (e.g., sepsis). At least a portion of the method may be performed by a computer system.

At block 1410, the biological sample is obtained from the subject. As examples, the biological sample can be blood, plasma, serum, urine, saliva, sweat, tears, and sputum, as well as other examples provided herein. In some embodiments (e.g., for blood), the biological sample can be purified for the mixture of cell-free DNA molecules, e.g., centrifuging blood to obtain plasma. The biological sample includes a mixture of cell-free DNA molecules derived from a subject and microbes.

At block 1420, the mixture of cell-free DNA molecules of the biological sample are analyzed to obtain sequence reads. The sequencing may be performed in a variety of ways, e.g., using massively parallel sequencing or next-generation sequencing, using single molecule sequencing, and/or using double- or single-stranded DNA sequencing library preparation protocols. In some instances, the biological sample is enriched for DNA molecules from the microbes can include using capture probes that bind to a portion of, or an entire genome of, the microbes. Block 1420 may be performed in a similar manner as block 620 of FIG. 6.

At block 1430, the sequence reads that were obtained from the sequencing of the mixture of cell free DNA molecules are received. The sequence reads include ending sequences corresponding to ends of the cell-free DNA molecules. Block 1430 may be performed in a similar manner as block 630 of FIG. 6.

At block 1440, one or more sequence reads that align to the one or more reference microbe genomes are determined from the obtained sequence reads. Each of the reference microbe genomes corresponds to a particular species of microbes. The particular microbe species can correspond to a species of one of the microbial genera consisting of Bacteroides, Klebsiella, Escherichia, Enterobacter, Citrobacter, Aeromonas, Mycobacterium, Candida, Prevotella, Streptococcus, and Orientia. Block 1440 may be performed in a similar manner as block 640 of FIG. 6.

In some embodiments, one or more sequence reads that include both ends of the nucleic acid fragment can be received. Thus, a plurality of sequence reads can be obtained from a sequencing of the mixture of cell free nucleic acid molecules. The one or more sequence reads can be aligned to the reference genome to obtain one or more aligned locations. The one or more aligned locations can be used to determine the size of the nucleic acid fragment.

Before the sequencing is performed, one or more assays can be used to determine whether a sufficient amount of the microbes is detected, and therefore warrants the sequencing to be performed. In some implementations, real-time polymerase chain reaction (PCR) can be performed using the biological sample or a different biological sample obtained from the subject contemporaneously (e.g., same clinical visit) as the biological sample. The real-time PCR can provide a quantity of nucleic acid molecules from the microbes using techniques described herein or known to one skilled in the art, e.g., using Ct values. The quantity can be compared to a quantity threshold. When the quantity is above the quantity threshold, the sequencing can be performed, thereby not wasting resources sequencing samples that do not have a sufficient quantity of microbial nucleic acids to warrant the more accurate technique. In some embodiments, digital PCR could be used instead of sequencing. The capture probes can be used with corresponding primers in performing the count of sequence reads.

In some instances, the aligned sequence reads are determined by filtering out sequences that align to a human reference genome. For example, the sequence reads can be aligned to the human reference genome. A subset of sequence reads aligning to the human reference genome can be filtered out. The remaining sequence reads can then be realigned to the one or more reference microbe genomes. A sequence read of the remaining sequence reads that aligns to a reference microbe genome of a particular microbe species (e.g., B. fragilis) can be determined the sequence read as being associated the particular microbe species.

The following blocks 1450 to 1470 can be iterated for each reference microbe genome of the one or more reference microbe genomes. For example, the level of infection for a particular microbial species can be determined based on analyzing sequence reads that align to the a corresponding reference microbe genome. In some instances, the level of infection can be determined across multiple microbial species by iterating blocks 1450 to 1470 through multiple reference microbe genomes.

In some instances, blocks 1450 to 1470 are performed once for all of the one or more reference microbe genomes, in which the level of infection would be based on microbial species that correspond to the one or more reference microbe genomes.

At block 1450, a set of sequence reads are identified from the one or more aligned sequence reads. In some embodiments, each sequence read of the set of the sequence reads includes an ending sequence corresponding to a sequence end signature of a plurality of sequence end signatures. Block 1450 may be performed in a similar manner as block 1050 of FIG. 10.

In some instances, a sequence end signature of the plurality of sequence end signatures corresponds to a 4-mer end motif (e.g., CCGA-end). The plurality of sequence end signatures can include 256 4-mer sequence end signatures. In effect, the set of sequence reads can include subsets of sequence reads, in which each subset corresponds to a respective sequence end signature. The skilled person will appreciate different types of sequence end signatures that can be used to identify the set of sequence reads (e.g., 1-mer, 2-mer, 3-mer, 5-mer, 6-mer). In some instances, the plurality of sequence end signatures corresponds to end motifs having various lengths.

At block 1460, a plurality of parameters are determined for of the set of the sequence reads. The plurality of parameters can be utilized to determine whether a microorganism associated with the set of sequence reads is pathogenic. In some embodiments, each parameter corresponds to a ratio of a plurality of O/E ratios determined for the set of sequence reads (e.g., via principal component analysis). Each O/E ratio of the plurality of O/E ratios can be derived based on: (i) a frequency of a subset of the set of sequence reads that correspond to a respective sequence end signature of the plurality of sequence end signatures; and (ii) a corresponding expected frequency for the respective sequence end signature.

At block 1470, the plurality of parameters are processed by a machine-learning model to generate an output classification. The output classification can identify a level of infection for the biological sample. A training set can be developed from samples having infection-causing microbial cell-free DNA. The training of the machine learning model can provide a formulation for how the output classification is determined. The machine-learning model includes one of logistic regression, support vector machines (SVM), decision tree, naïve Bayes classification, clustering algorithm, principal component analysis, singular value decomposition (SVD), t-distributed stochastic neighbor embedding (tSNE), artificial neural network, or ensemble methods.

In some instances, the output classification also identifies that the biological sample includes infection-causing pathogen-derived microbial DNA and further provides one or more genera or species of microbes (e.g., Bacteroides) that are associated with the infection-causing pathogen-derived microbial DNA.

As an illustrative example, the plurality of parameters are processed by using principal component analysis (PCA) to generate the output classification for the biological sample. The output classification can be used to determine the level of infection for the subject. In another example, the plurality of parameters are processed by using a support vector machine (SVM) to generate the output classification for the biological sample. The output classification can be used to determine the level of infection for the subject.

The level of infection can indicate the presence, absence, or an amount of infective microorganisms in the biological sample. In some instances, the level of infection indicates infection at a particular site, in which the infection site originates from biliary, lung, liver, abdomen, bowel, and/or urinary tract.

### VII. Microbial DNA Fragment Analysis of Maternal DNA

In some instances, microbial cell-free DNA molecules obtained from a sample of a pregnant female can be analyzed using end signature and size, to predict a level of infection for the pregnant female. The prediction of infection can be performed for the pregnant females, to identify certain types of microbes (e.g., Streptococcus) that are known to cause preterm labor.

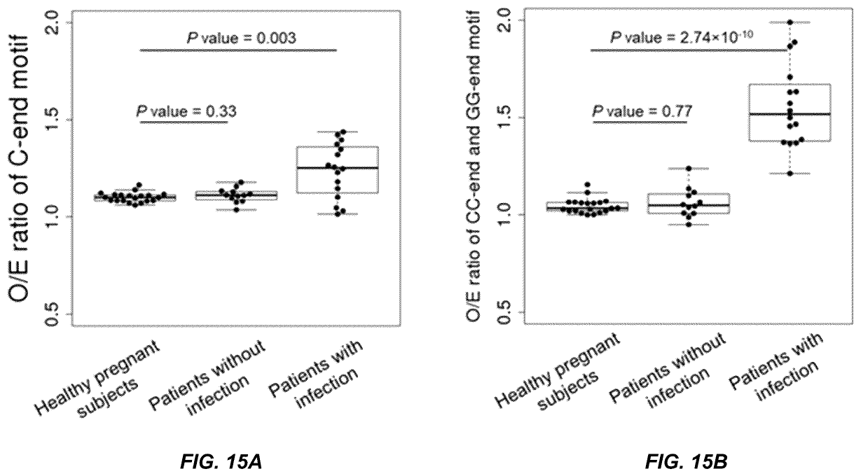

We sequenced 20 pregnant subjects with a median of 182 million of sequenced paired-end reads (range: 152-240 million). FIGS. 14A-B show diagrams that identify end signatures of microbial DNA fragments in pregnant subjects, according to some embodiments. FIG. 15A shows a boxplot that identifies O/E ratio of C-end motif of microbial DNA in healthy pregnant subjects and patients with/without infection. FIG. 15B shows a boxplot that identifies O/E ratio of CC-end and GG-end motif of microbial DNA in healthy pregnant subjects and patients with/without infection.

The boxplots in FIGS. 15A-B show that the O/E ratio of C-end motif or O/E ratio of CC- and GG-end motif of microbial DNA reads detected in healthy pregnant subjects were significantly lower than those from patients with infection. Therefore, such approach of end motif analysis could be used to differentiate infection-causing pathogen-derived microbial DNA and contaminant microbial DNA in pregnancy. The detection of infection-causing pathogen-derived microbial DNA can include identifying whether the infection-causing pathogen-derived microbial DNA correspond to certain types of microbes (e.g., streptococcus) that are known to cause preterm labor. In some instances, the classification of a level of infection includes an infection conducive to preterm labor. The profiling of microbiome during pregnancy or pregnancy with complications would be clinically relevant, such as but not limited to plasma microbial cell-free DNA analyses for preeclampsia, premature birth, pregnancy with bacterial infections (e.g. bacterial vaginosis).

### VIII. Treatment

Embodiments may further include treating a subject after determining a classification of a level of infection for the subject. For example, treatment can be provided according to a predicted amount of pathogens in the biological sample of the subject. In some instances, the treatment is provided based on a type of tissue at which the infection has occurred. The tissue can be used to guide a surgery or any other form of treatment. And, the level of infection can be used to determine how aggressive to be with any type of treatment, which may also be determined based on the level of pathology. For example, sepsis may be treated by an antibiotic treatment and blood pressure support drugs. In some embodiments, the more the value of a parameter (e.g., amount or size) exceeds the reference value, the more aggressive the treatment may be.

Example treatments for treating the microbial infection includes, but are not limited to the following: antibiotics or antibacterials; antivirals; antiparasitic agents; and antifungals. In some instances, different types of drugs and treatments are provided based on a type of microbe species identified from the subject. For example, if Mycobacterium tuberculosis is found in the subject, drugs such as Isoniazid (INH), rifampin (RIF), rifabutin, rifapentine (RPT), pyrazinamide (PZA), or any fluoroquinolone can be provided. In another example, if Clostridium botulinum bacteria is identified in the subject, antitoxins can be provided.

### IX. Example Systems

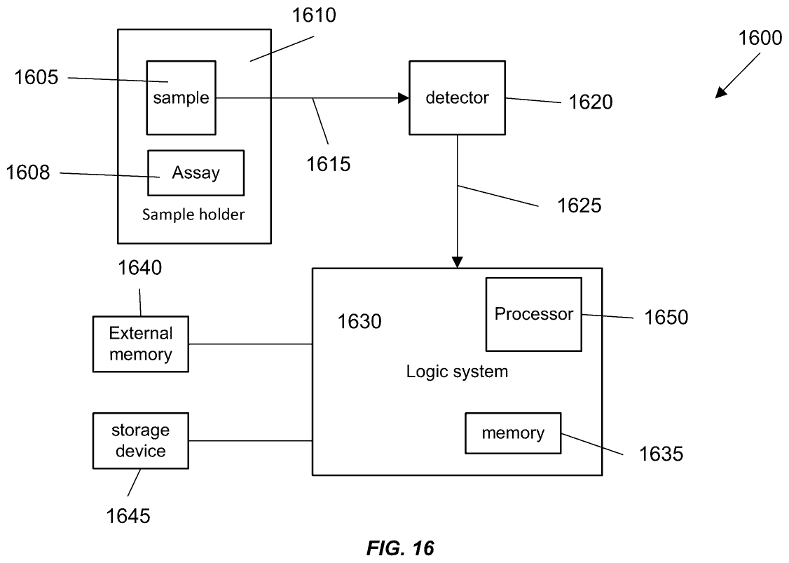

FIG. 16 illustrates a measurement system 1600 according to an embodiment of the present invention. The system as shown includes a sample 1605, such as cell-free DNA molecules within a sample holder 1610, where sample 1605 can be contacted with an assay 1608 to provide a signal of a physical characteristic 1615. An example of a sample holder can be a flow cell that includes probes and/or primers of an assay or a tube through which a droplet moves (with the droplet including the assay). Physical characteristic 1615 (e.g., a fluorescence intensity, a voltage, or a current), from the sample is detected by detector 1620. Detector 1620 can take a measurement at intervals (e.g., periodic intervals) to obtain data points that make up a data signal. In some instances, an analog-to-digital converter converts an analog signal from the detector into digital form at a plurality of times. Sample holder 1610 and detector 1620 can form an assay device, e.g., a sequencing device that performs sequencing according to embodiments described herein. A data signal 1625 is sent from detector 1620 to logic system 1630. Data signal 1625 may be stored in a local memory 1635, an external memory 1640, or a storage device 1645.

Logic system 1630 may be, or may include, a computer system, ASIC, microprocessor, etc. It may also include or be coupled with a display (e.g., monitor, LED display, etc.) and a user input device (e.g., mouse, keyboard, buttons, etc.). Logic system 1630 and the other components may be part of a stand-alone or network connected computer system, or they may be directly attached to or incorporated in a device (e.g., a sequencing device) that includes detector 1620 and/or sample holder 1610. Logic system 1630 may also include software that executes in a processor 1650. Logic system 1630 may include a computer readable medium storing instructions for controlling measurement system 1600 to perform any of the methods described herein. For example, logic system 1630 can provide commands to a system that includes sample holder 1610 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

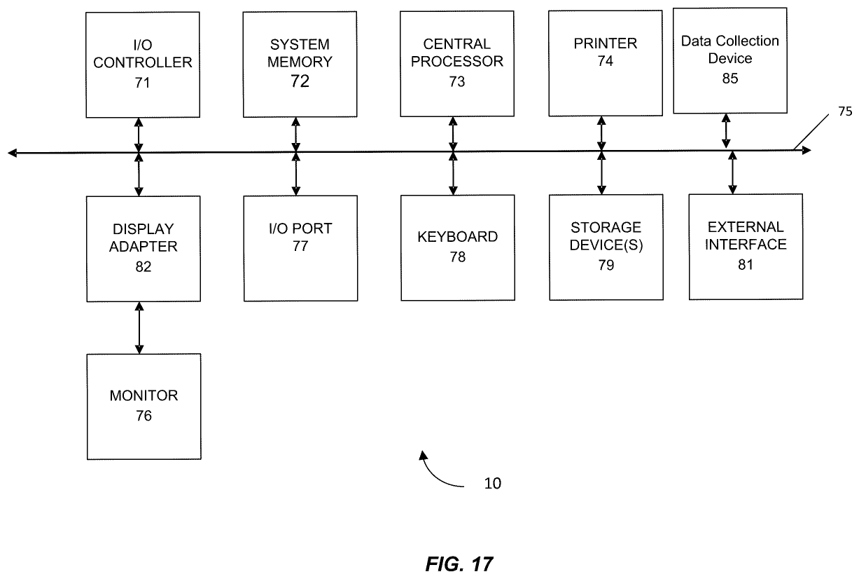

Any of the computer systems mentioned herein may utilize any suitable number of subsystems. Examples of such subsystems are shown in FIG. 17 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 17 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The section headings used herein are for organizational purposes only and are not to be construed as limiting the subject matter described.

It is to be understood that the methods described herein are not limited to the particular methodology, protocols, subjects, and sequencing techniques described herein and as such may vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention.

Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

All patents, patent applications, publications, and descriptions mentioned herein are incorporated by reference in their entirety for all purposes. None is admitted to be prior art.

What is claimed is:

1. A method of analyzing a biological sample to determine a level of infection in a subject, the biological sample including a mixture of cell-free DNA molecules from the subject and microbes, the method comprising:
for each of a set of cell-free DNA molecules in the biological sample:
measuring a size of the cell-free DNA molecule; and
determining that the cell-free DNA molecule is from one or more reference microbe genomes, each of the one or more reference microbe genomes corresponding to a particular species of microbes;
determining a statistical value of the measured sizes of the plurality of cell-free DNA molecules;
comparing the statistical value to a cutoff value; and
determining the level of infection in the subject based on the comparison.

2. The method of claim 1, wherein the statistical value is an average, mode, median, or mean of the measured sizes.

3. The method of claim 1, wherein the statistical value is a percentage of the set of cell-free DNA molecules that are below a size threshold.

4. The method of claim 3, wherein the cutoff value is a numerical value selected between 75 bp and 90 bp.

5. The method of claim 3, wherein the subject is determined to be positive for the infection when statistical value is above the cutoff value.

6. The method of claim 1, wherein determining that the set of cell-free DNA molecules are from one or more reference microbe genomes includes:
analyzing the mixture of cell-free DNA molecules to obtain sequence reads;
aligning the sequence reads to a reference human genome;
identifying one or more non-aligned sequence reads by filtering out, from the sequence reads, a plurality of sequence reads that align to the reference human genome;
realigning the one or more non-aligned sequence reads to the one or more reference microbe genomes to identify a set of sequence reads that correspond to microbial DNA molecules; and
identifying the set of cell-free DNA molecules based on the set of sequence reads that correspond to the microbial DNA molecules.

7. The method of claim 6, further comprising enriching the set of cell-free DNA molecules.

8. The method of claim 1, wherein the particular species of microbes is selected from a group of microbial genera consisting of Bacteroides, Klebsiella, Escherichia, Enterobacter, Citrobacter, Aeromonas, Mycobacterium, Candida, Prevotella, Streptococcus, and Orientia.

9. The method of claim 1, wherein measuring the size of the cell-free DNA molecule of the set of cell-free DNA molecules includes:
receiving one or more sequence reads that include both ends of the cell-free DNA molecule, thereby obtaining a plurality of sequence reads from a sequencing of the mixture of cell-free DNA molecules;
aligning the one or more sequence reads to the one or more reference microbe genomes to obtain one or more aligned locations; and
using the one or more aligned locations to determine the size of the cell-free DNA molecule.

10. The method of claim 9, further comprising:
performing sequencing of the mixture of cell-free DNA molecules to obtain the plurality of sequence reads.

11. The method of claim 10, further comprising:
performing real-time polymerase chain reaction (PCR) of the biological sample or a different biological sample obtained from the subject contemporaneously as the biological sample, thereby determining a quantity of DNA molecules from microbes;
comparing the quantity to a quantity threshold; and
when the quantity is above the quantity threshold, performing the sequencing of the mixture of cell-free DNA molecules.

12. The method of claim 10, wherein the sequencing includes random sequencing.

13. A method of analyzing a biological sample to determine a level of infection in a subject, the biological sample including a mixture of cell-free DNA molecules from the subject and microbes, the method comprising:
analyzing a plurality of cell-free DNA molecules from the biological sample to obtain sequence reads, wherein the sequence reads include ending sequences corresponding to ends of the plurality of cell-free DNA molecules;
aligning the sequence reads to one or more reference microbe genomes to identify aligned sequence reads, each of the one or more reference microbe genomes corresponding to a particular species of microbes;
identifying a set of the sequence reads from the aligned sequence reads, wherein each sequence read of the set of the sequence reads includes an ending sequence corresponding to a set of one or more sequence end signatures;
determining a parameter for the set of the sequence reads based at least in part on a first amount of the set of sequence reads; and
determining a classification of a level of infection using the parameter.

14. The method of claim 13, wherein the parameter is a frequency determined based on the first amount of the set of sequence reads.

15. The method of claim 13, wherein the parameter is a first ratio between: (i) a first observed frequency determined based on an amount of a first subset of the set of sequence reads, wherein the first subset of sequence reads include an ending sequence corresponding to a first sequence end signature of the set of one or more sequence end signatures; and (ii) a first expected frequency for the first sequence end signature.

16. The method of claim 15, wherein the parameter is a combined value determined based on the first ratio and a second ratio, wherein the second ratio is between: (i) a second observed frequency determined based on an amount of a second subset of the set of sequence reads, wherein the second subset of sequence reads include an ending sequence corresponding to a second sequence end signature of the set of one or more sequence end signatures; and (ii) a second expected frequency for the second sequence end signature.

17. The method of claim 15, wherein the parameter is a ratio determined based on the first ratio and a second ratio, wherein the second ratio is between: (i) a second observed frequency determined based on an amount of a second subset of the set of sequence reads, wherein the second subset of sequence reads include an ending sequence corresponding to a second sequence end signature of the set of one or more sequence end signatures; and (ii) a second expected frequency for the second sequence end signature.

18. The method of claim 13, wherein the determination of the classification of the level of infection is based on a comparison between the parameter and a reference value.

19. The method of claim 13, wherein the level of infection indicates a presence of sepsis.

20. The method of claim 13, further comprising:
for each of the plurality of cell-free DNA molecules in the biological sample:
measuring a size of the cell-free DNA molecule; and
determining that the cell-free DNA molecule is from the one or more reference microbe genomes;
determining a statistical value of the measured sizes of the plurality of cell-free DNA molecules;
comparing the statistical value to a cutoff value; and
further determining the level of infection in the subject based on the comparison.

21. The method of claim 13, wherein aligning the sequence reads to one or more reference microbe genomes includes:
aligning the sequence reads to a reference human genome;
identifying one or more non-aligned sequence reads by filtering out, from the sequence reads, a plurality of sequence reads that align to the reference human genome; and
realigning the one or more non-aligned sequence reads to the one or more reference microbe genomes to identify the aligned sequence reads.

22. The method of claim 21, further comprising enriching the set of sequence reads.

23. The method of claim 13, wherein determining the classification of the level of infection includes processing the first amount of the set of the sequence reads using a machine-learning model.

24. The method of claim 23, wherein the machine-learning model includes one of logistic regression, support vector machines (SVM), decision tree, naïve Bayes classification, clustering algorithm, principal component analysis, singular value decomposition (SVD), t-distributed stochastic neighbor embedding (tSNE), artificial neural network, or ensemble methods.

25. The method of claim 13, wherein the subject is a pregnant female, and wherein the classification of the level of infection includes an infection conducive to preterm labor.

26. A system for analyzing a biological sample to determine a level of infection in a subject, the biological sample including a mixture of cell-free DNA molecules from the subject and microbes, the system comprising:
a processor; and
a memory coupled to the processor, the memory storing instructions, which when executed by the processor, cause the processor to perform operations to:
for each of a set of cell-free DNA molecules in the biological sample:
measure a size of the cell-free DNA molecule; and
determine that the cell-free DNA molecule is from one or more reference microbe genomes, each of the one or more reference microbe genomes corresponding to a particular species of microbes;
determine a statistical value of the measured sizes of the plurality of cell-free DNA molecules;
compare the statistical value to a cutoff value; and
determine the level of infection in the subject based on the comparison.

27. A system of analyzing a biological sample to determine a level of infection in a subject, the biological sample including a mixture of cell-free DNA molecules from the subject and microbes, the system comprising:
a processor; and
a memory coupled to the processor, the memory storing instructions, which when executed by the processor, cause the processor to perform operations to:
analyze a plurality of cell-free DNA molecules from the biological sample to obtain sequence reads, wherein the sequence reads include ending sequences corresponding to ends of the plurality of cell-free DNA molecules;
align the sequence reads to one or more reference microbe genomes to identify aligned sequence reads, each of the one or more reference microbe genomes corresponding to a particular species of microbes;
identify a set of the sequence reads from the aligned sequence reads, wherein each sequence read of the set of the sequence reads includes an ending sequence corresponding to a set of one or more sequence end signatures;
determine a parameter for the set of the sequence reads based at least in part on a first amount of the set of sequence reads; and
determine a classification of a level of infection using the parameter.
